(19) United States
(12) Patent Application Publication
(10) Pub. No.: US 2017/0121268 A1
(43) Pub. Date: May 4, 2017

(54) PPAR MODULATORS

(71) Applicants: Luiz Antonio Soares ROMEIRO, Brasília (BR); Carolyn CUMMINS, Toronto (CA); Lilia MAGOMEDOVA, Richmond Hill (CA); THE GOVERNING COUNCIL OF THE UNIVERSITY OF TORONTO, Toronto (CA); UNIÃO BRASILIENSE DE EDUCAÇÃO E CULTURA - UBEC, Brasília (BR); FUNDAÇAO UNIVERSIDADE DE BRASÍLIA, Brasília (BR)

(72) Inventors: Luiz Antonio Soares Romeiro, Lago Norte (BR); Carolyn Cummins, Toronto (CA); Lilia Magomedova, Richmond Hill (CA)

(21) Appl. No.: 15/313,828

(22) PCT Filed: May 23, 2014
(86) PCT No.: PCT/CA2014/000448

§ 371 Date: Nov. 23, 2016

(51) Int. Cl. C07C 69/734; C07C 69/708; C07C 59/64

(52) U.S. Cl. CPC C07C 69/734; C07C 59/64; C07C 69/708

(57) ABSTRACT

The present application relates to amorfrutin analogs and uses as PPAR modulators for the treatment of metabolic syndrome, obesity, hyperlipidemia, elevated fasting blood glucose, elevated blood pressure, low HDL cholesterol, type 2 diabetes, cardiovascular disease, a neurodegenerative disease, malaria or irritable bowel syndrome.

### FIELD

The present application is directed to compounds of Formula (I), (II) and (III), which are multi-specific peroxisome proliferator-activated receptor (PPARα and PPARγ) ligands.

### INTRODUCTION

Metabolic syndrome refers to a constellation of medical conditions associated with increased risk for development of type 2 diabetes and cardiovascular disease. The co-occurrence of central obesity along with two out of the following four conditions defines the presence of metabolic syndrome: hyperlipidemia, elevated fasting blood glucose, elevated blood pressure and low HDL cholesterol (1). Approximately 25% of the world's population is estimated to have metabolic syndrome (1).

Primary treatment for a diagnosis of metabolic syndrome is lifestyle intervention including decreased caloric intake and increased exercise (2). Following that, second line treatment frequently involves the use of pharmacologic agents that target the PPAR (peroxisome proliferator activated receptor) subclass of nuclear receptor proteins (2-4). Anti-hyperlipidemic agents, known as the fibrate class of drugs, target PPARα and are effective at lowering hypertriglyceridemia and increasing HDL (5). In contrast, the insulin sensitizing thiazolidinedione class of drugs (TZDs) target PPARγ, a receptor important in the control of glucose homeostasis (3).

The PPAR receptors are transcription factors that act as heterodimers with the retinoid x receptor (RXR) when complexed on DNA. Upon ligand activation, there is a conformational change in the three-dimensional structure of the receptor that promotes interaction with other proteins such as co-regulators (activators or repressors) thereby modulating the recruitment of basal transcriptional machinery, and influencing gene expression.

PPARα agonists (i.e., bezafibrate, fenofibrate, gemfibrozil, clofibrate) exert their hypolipidemic effects by coordinately increasing the transcription of genes important in liver fatty acid uptake and fatty acid oxidation. Liver activation of PPARα increases the expression of the fatty acid transport protein CD36 and the intracellular fatty acid binding protein FABP1, which together contribute to an increased flux of fatty acids into the liver. Pyruvate dehydrogenase kinase 4 (PDK4), another gene induced by PPARα, provides a critical signal that directs the cell to switch from glucose utilization toward fatty acid utilization. Finally, several genes in the fatty acid β-oxidation pathway are directly induced by PPARα. These include the mitochondrial transporter carnitine palmitoyl transferase protein (CPT1) that helps transport acyl-CoA-modified fatty acids to the mitochondria for β-oxidation; medium chain acyl-coenzyme A dehydrogenase (ACADM) that promotes the breakdown of medium chain fatty acids; and peroxisomal acyl-coenzyme A oxidase 1 (ACOX1) that catalyzes the first enzyme in the β-oxidation pathway.

PPARα activation also results in increased circulating HDL (the ‘good cholesterol’ lipoprotein that promotes reverse cholesterol transport) via increased ApoA1 secretion. Decreased inflammation has also been found to occur with PPARα activation (6). PPARα increases the expression of IκBα which promotes NF-κB degradation thereby reducing pro-inflammatory gene expression (7).

PPARα has recently been shown to activate the starvation hormone FGF21 (8,9). FGF21 is currently being developed as an anti-obesity therapy because it has been shown in animal studies that when given in pharmacologic doses that it increases insulin sensitization, increases energy expenditure, and decreases body weight (10-12). FGF21 acts by increasing fatty acid oxidation through the induction of the nuclear receptor coactivator PGC1α (peroxisome proliferator-activated receptor-γ coactivator 1α) (13). FGF21 also promotes insulin sensitization via increasing glucose uptake in adipose and muscle, suppressing hepatic glucose production, and decreasing adipose tissue lipolysis (14-17). Finally, FGF21 was recently shown to increase energy expenditure by signaling to receptors in the brain that control sympathetic input to thermogenic brown adipose tissue (18-20). Taken together, upregulation of PPARα target genes results in improved systemic lipid homeostasis that includes reduced circulating plasma triglycerides, decreased LDL cholesterol, and increased HDL cholesterol.

PPARγ agonists (i.e., pioglitazone and rosiglitazone) exert their insulin sensitization effects via several mechanisms (21-23). TZDs lower plasma free fatty acids (FFAs) by coordinating their uptake into liver and fat to be oxidized (22,24). Fat uptake in adipose is mediated by several factors including lipoprotein lipase (LPL), which hydrolyzes circulating triglycerides; the fatty acid uptake transporter CD36, and the fatty acid binding protein aP2, also known as fatty acid binding protein 4. (25) TZDs also increase the expression of the glucose transporter 4 (GLUT4) in adipose tissue to promote glucose uptake, and uncoupling protein 2 (UCP2) to increase energy expenditure.

PPARγ activation by TZDs also increases the expression of several adipokines that influence insulin sensitivity including adiponectin, resistin and leptin (26). Adiponectin is a fat-derived hormone secreted into circulation that increases insulin sensitivity by activating AMPK in muscle and liver (27-29). PPARγ is required for adipogenesis and for the maintenance of fully differentiated fat cells. As such, TZDs have also been shown to increase the number of small adipocytes, which have been shown are generally more insulin sensitive than large adipocytes (25,30). At the same time, PPARγ activation in the macrophage is anti-inflammatory and contributes to the maintenance of the anti-inflammatory ‘M2’ macrophages (31,32).

Unfortunately, the development of major side effects remains a serious limitation for the therapeutic use of currently prescribed PPARγ agonists. Common side effects of the PPARγ agonists (TZDs) include weight gain, increased fat mass, increased bone fracture rate, fluid retention, macular edema, congestive heart failure and myocardial infarction (33). Studies have shown that women on rosiglitazone or pioglitazone have a two-fold increased rate of distal bone fractures (34,35). Additionally, the incidence of fluid retention and peripheral edema in patients taking TZD is approximately 5% (34,36). PPARα agonists also suffer from undesired side effects including increased liver enzymes, increased homocysteine and increased creatinine. These effects preclude the use of PPARα agonists in patients with severe renal dysfunction, liver disease and gallbladder disease (37).

An improved safety profile of a drug combining the best features of both PPARα and PPARγ activation would be an attractive therapeutic modality for the treatment of metabolic syndrome, type 2 diabetes, diabetic dyslipidemia, hyperlipidemia, atherosclerosis and microvascular complications of diabetes (38). Indeed, partial PPARγ agonists or weak PPARγ ligands have previously been shown in mice to have insulin sensitizing effects without the side effect of weight gain (39,40). Compounds with these characteristics are termed selective PPAR modulators (SPPARMs). SPPARMs are characterized by their tissue-selective and/or gene-selective activities. This can be achieved through partial agonism of the receptor or by reduced potency relative to potent full PPAR agonists. The key feature is that these compounds allow the separation of the negative effects of PPAR activation from the beneficial (therapeutic) effects. The difference in response between highly potent full agonists and SPPARMs is thought to arise because a distinct constellation of co-activators are recruited upon ligand binding which influences the downstream physiologic response (40a).

PPARγ agonists are also useful for the treatment of several other diseases such as malaria (41-44) and Alzheimer's disease (45). In both conditions, the anti-inflammatory actions of TZDs contribute, at least in part, to the improvement with drug treatment (41,43,46-48). In the context of malaria treatment, activation of PPARγ induces the expression of CD36 in macrophages, which strongly enhances parasite clearance from peripheral blood (44,49-51). A randomized, doubleblind, placebo-controlled trial of standard anti-malarial treatment in combination with rosiglitazone, found that patients treated with rosiglitazone exhibited enhanced parasite clearance and reduced inflammatory cytokines in plasma compared to patients that received placebo (43).

Over the past several years, brain insulin resistance has been postulated to contribute to the pathogenesis of Alzheimer's disease (AD) (52,53). Indeed, patients with type 2 diabetes have a 1.9-times greater risk of developing AD compared to normoglycemic patients (53a). Likewise, it was shown that in one study of AD patients, 80% of participants had impaired fasting blood glucose or type 2 diabetes (53b). Pathologically, AD is characterized by the accumulation and deposition of extracellular amyloid β plaques, neurofibrillary tangle formation, chronic neuroinflammation and intraneuronal tau protein accumulation (54). Insulin resistance further increases cerebral tau deposition (54a).

According to the amyloid hypothesis of AD, various amyloid species contribute to disease. Amyloid β(Aβ) peptide oligomers and amyloid fibrils are formed from Aβ42, a proteolytic fragment resulting from sequential cleavage of the amyloid precursor protein (APP) by the aspartyl proteases, β-secretase and γ-secretase. Inhibition of γ-secretase is currently one of the most promising targets for the treatment of AD. In addition, soluble Aβ oligomers bind and activate the synaptic signaling pathways that result in hyperphosphorylation of tau protein, increased oxidative stress, and the deterioration and loss of synapses (54).

There are several mechanisms by which PPARγ activation is beneficial in the context of Alzheimer's disease (AD) (55). Activation of PPARγ minimizes neuroinflammation and induces the expression of CD36 in microglia. CD36 has been found to promote Aβ clearance by microglia, thereby contributing to the removal of Aβ (55a). Furthermore, PPARγ induces the expression of ApoE, a secreted protein that promotes the proteolytic degradation of soluble Aβ peptide (55b). The administration of an RXR agonist (RXR is the heterodimer partner for PPAR) to mouse models of AD were shown to reduce Aβ levels and promote cognitive improvement via a mechanism that was dependent on ApoE (56). Finally, PPARγ activation has been shown to modulate γ-secretase function by transcriptionally inhibiting the expression of β-secretase (BACE1) (57). Consistent with these mechanisms, several animal models of AD have shown significant improvements in cognitive function with rosiglitazone and pioglitazone treatment (58-61). Preliminary studies in humans have confirmed the beneficial effect of pioglitazone and rosiglitazone for the treatment of mild to moderate cognitive impairment in AD (62-64).

### SUMMARY

The present disclosure relates to compounds, and uses of compounds, which are PPAR modulators (SPPARMs). In particular, in one embodiment of the disclosure, the compounds are selective PPAR modulators for PPARα and PPARγ and act as dual agonists.

In one embodiment, the present disclosure includes compounds of the Formula (I)

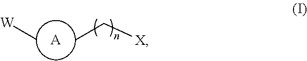

wherein

### Ring A is

(i) optionally substituted (C₆-C₁₀)-aryl, or

(ii) optionally substituted (C₅-C₁₀)-heteroaryl,

in which the optional substituents are selected from one to four of halo, OH, O—(C₁-C₆)-alkyl, (C₁-C₆)-alkyl, —C(O)OH, —OC(O)—(C₁-C₆)-alkyl or —C(O)O—(C₁-C₆)-alkyl;

### X is

(i) R′,

(ii) —OR′,

(iii) —NR′R″,

(iv) —C(O)O—R′,

(v) —C(O)—R′, or

(vi) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl,

### W is

(i) —O—(C₁-C₆)alkyl, or

(ii) (C₂-C₆)-alkylene-C(O)OR, wherein
(ii.a) wherein at least one carbon of the (C₂-C₆)-alkylene group is substituted with R₁ and/or R₂; and
(ii.b) wherein at least one carbon of the (C₂-C₆)-alkylene group is replaced with an oxygen atom;
   R₁ and R₂ are each independently or simultaneously

(i) H,

(ii) (C₁-C₈)-alkyl,

(iii) (C₃-C₈)-alkyl, or

(iv) R₁ and R₂ together form a (C₃-C₈)-alkyl ring,

R is H or (C₁-C₈)-alkyl, and
n is 3, 4, 5, 6, 7, 8, 9, or 10,
or a pharmaceutically acceptable salt, solvate, prodrug and/or stereoisomer thereof,
and wherein the following compounds are excluded from the Formula (I)

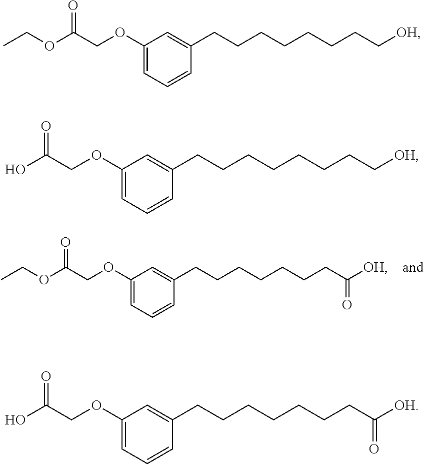 [SMILES: CCOC(=O)COc1cccc(CCCCCCCCO)c1 ; O=C(O)COc1cccc(CCCCCCCCO)c1 ; CCOC(=O)COc1cccc(CCCCCCCC(=O)O)c1 ; O=C(O)CCCCCCCc1cccc(OCC(=O)O)c1]

In another embodiment, the compounds of the Formula (I) are compounds of the Formula (Ia)

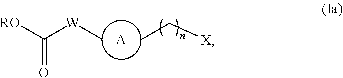

wherein

### Ring A is

(i) optionally substituted (C₆-C₁₀)-aryl, or
(ii) optionally substituted (C₅-C₁₀)-heteroaryl,
   in which the optional substituents are selected from one to four of halo, OH, O—(C₁-C₆)-alkyl, (C₁-C₆)-alkyl, —C(O)OH, or —C(O)O—(C₁-C₆)-alkyl;

### X is

(i) R′,

(ii) —OR′,

(iii) —NR′R″,

(iv) —C(O)O—R′,

(v) —C(O)—R′, or

(vi) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl,

W is (C₂-C₆)-alkylene-C(O)OR, wherein
(ii.a) wherein at least one carbon of the (C₂-C₆)-alkylene group is substituted with R₁ and/or R₂; and
(ii.b) wherein at least one carbon of the (C₂-C₆)-alkylene group is replaced with an oxygen atom;
   R₁ and R₂ are each independently or simultaneously

(i) (C₁-C₈)-alkyl,

(ii) (C₃-C₈)-alkyl, or

(iii) R₁ and R₂ together form a (C₃-C₈)-alkyl ring,

R is H or (C₁-C₈)-alkyl, and
n is 3, 4, 5, 6, 7, 8, 9, or 10,
or a pharmaceutically acceptable salt, solvate, prodrug and/or stereoisomer thereof.

The present disclosure also includes a use of a compound of the Formula (II) as a PPAR modulator, for example a selective PPAR modulator, wherein the compound of the Formula (II) has the following structure

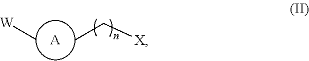

wherein

### Ring A is

(i) optionally substituted (C₆-C₁₀)-aryl, or

(ii) optionally substituted (C₅-C₁₀)-heteroaryl,

in which the optional substituents are selected from one to four of halo, OH, O—(C₁-C₆)-alkyl, (C₁-C₆)-alkyl, —C(O)OH, or —C(O)O—(C₁-C₆)-alkyl;

### X is

(i) R′,

(ii) —OR′,

(iii) —NR′R″,

(iv) —C(O)O—R′,

(v) —C(O)—R′, or

(vi) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl,

### W is

(i) —O—(C₁-C₆)alkyl, or

(ii) (C₂-C₆)-alkylene-C(O)OR, wherein
(ii.a) at least one carbon of the (C₂-C₆)-alkylene group is substituted with R₁ and/or R₂; and
(ii.b) at least one carbon of the (C₂-C₆)-alkylene group is replaced with an oxygen atom;
   R₁ and R₂ are each independently or simultaneously

(i) H,

(ii) (C₁-C₈)-alkyl,

(iii) (C₃-C₈)-alkyl, or

(iv) R₁ and R₂ together form a (C₃-C₈)-alkyl ring,

R is H or (C₁-C₈)-alkyl, and
n is 3, 4, 5, 6, 7, 8, 9, or 10,
or a pharmaceutically acceptable salt, solvate, prodrug and/or stereoisomer thereof.

The present disclosure also includes a use of a compound of the Formula (III) as a PPAR modulator, for example a selective PPAR modulator, wherein the compound of the Formula (III) has the following structure

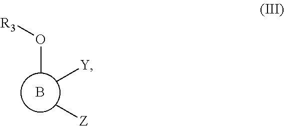

wherein

### Ring B is

(i) optionally substituted (C₆-C₁₀)-aryl, or

(ii) optionally substituted (C₆-C₁₀)-heteroaryl,

in which the optional substituents are selected from one to four of OH, O—(C₁-C₆)-alkyl, (C₁-C₆)-alkyl, —C(O)OH, or —C(O)O—(C₁-C₆)-alkyl; R₃ is

(i) H,

(ii) (C₁-C₆)-alkyl,

(iii) —C(═O)—(C₁-C₆)-alkyl,

(iv) —(C₁-C₆)-alkylene-C(═O)—O—R₄, wherein the (C₁-C₆)-alkylene moiety is optionally substituted by one or more of (C₁-C₆)-alkyl;

### R₄ is

(i) H, or

(ii) (C₁-C₆)-alkyl

### Y is

(i) H, or

(ii) —C(O)OH;

Z is (C₁₀-C₂₀)alkyl, having 0-3 unsaturated bonds,
or a pharmaceutically acceptable salt, solvate, prodrug and/or stereoisomer thereof.

The present disclosure also includes pharmaceutical compositions comprising compounds of the Formula (I), (II) and/or (Ill) and pharmaceutically acceptable excipients, carriers and/or additives.

In one embodiment, the compounds of the Formula (I), (II) and/or (III) may be synthesized from compounds present in cashew nut shell oil. In one embodiment, the compounds of the Formula (I), (II) and/or (III) are synthesized in high yield, using environmentally friendly chemistry, from a low-cost byproduct of cashew nut processing, which includes for example, anacardic acids, cardol and cardanol.

The present disclosure also includes a method of treating or preventing a disease or condition for which PPAR modulation provides a therapeutic benefit, comprising administering an effective amount of a compound of the Formula (I), (II) and/or (III) to a patient in need thereof.

The present disclosure also includes a use of a compound of the Formula (I), (II) and/or (III), for treating or preventing a disease or condition for which PPAR modulation provides a therapeutic benefit.

Other features and advantages of the present application will become apparent from the following detailed description. It should be understood, however, that the detailed description and the specific examples while indicating preferred embodiments of the application are given by way of illustration only, since various changes and modifications within the spirit and scope of the application will become apparent to those skilled in the art from this detailed description.

### DRAWINGS

The disclosure will now be described in greater detail with reference to the following drawings in which:

FIG. 1 is a graph showing the activity of compounds of the disclosure in a luciferase reporter screen for A) PPARα and B) PPARγ;

FIG. 2 is a dose response curve showing the activity of compounds of the disclosure;

FIG. 3 is a functional analysis in primary hepatocytes of compounds of the disclosure;

FIG. 4A is a functional analysis in a 3T3-L1 differentiation assay of compounds of the disclosure; FIG. 4B shows Oil red O staining in adipocytes after treatment with compounds of the disclosure;

FIG. 5 is a graph showing the activity of compounds of the disclosure in a second luciferase reporter screen for A) PPARα and B) PPARγ;

FIG. 6 is a second dose response curve showing the activity of compounds of the disclosure;

FIG. 7 is a second functional analysis in primary hepatocytes of compounds of the disclosure;

FIG. 8A is a second functional analysis in a 3T3-L1 differentiation assay of compounds of the disclosure; FIG. 8B shows a second Oil red O staining in adipocytes after treatment with compounds of the disclosure;

FIG. 9 is a graph showing the activity of compounds of the disclosure in a third luciferase reporter screen for A) PPARα and B) PPARγ;

FIG. 10 is a third dose response curve showing the activity of compounds of the disclosure;

FIG. 11 is a third functional analysis in primary hepatocytes of compounds of the disclosure; and

FIG. 12A is a third functional analysis in a 3T3-L1 differentiation assay of compounds of the disclosure; FIG. 12B shows a third Oil red O staining in adipocytes after treatment with compounds of the disclosure.

### DESCRIPTION OF VARIOUS EMBODIMENTS

### Definitions

The term “(C₁-Cₚ)-alkyl” as used herein means straight and/or branched chain, saturated alkyl radicals containing from one to “p” carbon atoms and includes (depending on the identity of p) methyl, ethyl, propyl, isopropyl, n-butyl, s-butyl, isobutyl, t-butyl, 2,2-dimethylbutyl, n-pentyl, 2-methylpentyl, 3-methylpentyl, 4-methylpentyl, n-hexyl and the like, where the variable p is an integer representing the largest number of carbon atoms in the alkyl radical.

The term “C₃₋ₚcycloalkyl” as used herein means a monocyclic, bicyclic or tricyclic saturated carbocylic group containing from three to “p′” carbon atoms and includes (depending on the identity of p) cyclopropyl, cyclobutyl, cyclopentyl, cyclodecyl and the like, where the variable p′ is an integer representing the largest number of carbon atoms in the cycloalkyl radical.

The term “heteroaryl” as used herein refers to aromatic cyclic or polycyclic ring systems having at least one heteroatom chosen from N, O and S and at least one aromatic ring. Examples of heteroaryl groups include, without limitation, furyl, thienyl, pyridyl, quinolinyl, isoquinolinyl, indolyl, isoindolyl, triazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl and quinazolinyl, among others.

The term “aryl” as used herein refers to cyclic groups that contain at least one aromatic ring, for example a single ring (e.g. phenyl) or multiple condensed rings (e.g. naphthyl). In an embodiment of the present disclosure, the aryl group contains 6, 9 or 10 atoms such as phenyl, naphthyl, indanyl, anthracenyl, 1,2-dihydronaphthyl, 1,2,3,4-tetrahydronaphthyl, fluorenyl, indanyl, indenyl and the like.

The suffix “ene” added on to any of the above groups means that the group is divalent, i.e. inserted between two other groups.

The term “halo” as used herein refers to a halogen atom and includes fluorine (F), chlorine (Cl), bromine (Br) and iodine (I).

The term “selective PPAR modulator” as used herein refers to compounds that have differential activation profiles towards heterologously expressed PPARα or PPARγ (i.e., partial or weak PPAR activity) or differential activation of PPAR target genes, when compared to highly potent standard full agonists of PPARα or PPARγ, for example GW7648 (for PPARα) and rosiglitazone (for PPARγ).

The term “pharmaceutically acceptable salt” refers, for example, to a salt that retains the desired biological activity of a compound of the present disclosure and does not impart undesired toxicological effects thereto; and may refer to an acid addition salt or a base addition salt.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

In embodiments of the present disclosure, the compounds may have an asymmetric center. These compounds exist as enantiomers. Where compounds possess more than one asymmetric center, they may exist as diastereomers. It is to be understood that all such isomers and mixtures thereof in any proportion are encompassed within the scope of the present disclosure. It is to be further understood that while the stereochemistry of the compounds may be as shown in any given compound listed herein, such compounds may also contain certain amounts (e.g. less than 20%, suitably less than 10%, more suitably less than 5%) of compounds of the disclosure having alternate stereochemistry. For example, compounds of the disclosure that are shown without any stereochemical designations are understood to be racemic mixtures (i.e. contain an equal amount of each possible enantiomer or diastereomer). However, it is to be understood that all enantiomers and diastereomers are included within the scope of the present disclosure, including mixtures thereof in any proportion.

The term “effective amount” or “therapeutically effective amount” as used herein means an amount effective, at dosages and for periods of time necessary to achieve the desired result. For example in the context of treating a subject with malaria, an effective amount is an amount that, for example, reduces the amount of parasite in the blood of a subject. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art.

As used herein, the term “prodrug” refers to a substance that is prepared in an inactive form that is converted to an active form (i.e., drug) within the body or cells thereof by the action of, for example, endogenous enzymes or other chemicals and/or conditions. Prodrug derivatives of the compounds of Formula (I), (II) or (III), or pharmaceutically acceptable salts or solvates thereof, can be prepared by methods known to those of ordinary skill in the art, and include esters of any free hydroxyl or carboxyl moieties of the compounds

Although the disclosure has been described in conjunction with specific embodiments thereof, if is evident that many alternatives, modifications and variations will be apparent to those skilled in the art. Accordingly, it is intended to embrace all such alternatives, modifications and variations that fall within the spirit and broad scope of the appended claims. In addition, citation or identification of any reference in this application shall not be construed as an admission that such reference is available as prior art to the present disclosure.

The operation of the disclosure is illustrated by the following representative examples. As is apparent to those skilled in the art, many of the details of the examples may be changed while still practicing the disclosure described herein.

### COMPOUNDS OF THE DISCLOSURE

The present disclosure relates to compounds which are PPAR modulator, and in particular, selective PPAR modulators (SPPARMs). In particular, in one embodiment of the disclosure, the compounds are selective PPAR modulators for PPARα and PPARγ and act as dual agonists.

In one embodiment, the present disclosure includes compounds of the Formula (I)

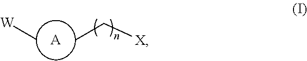

wherein

### Ring A is

(i) optionally substituted (C₆-C₁₀)-aryl, or

(ii) optionally substituted (C₆-C₁₀)-heteroaryl, in which the optional substituents are selected from one to four of halo, OH, O—(C₁-C₆)-alkyl, (C₁-C₆)-alkyl, —C(O)OH, or —C(O)O—(C₁-C₆)-alkyl;

### X is

(i) R′,

(ii) —OR′,

(iii) —NR′R″,

(iv) —C(O)O—R′,

(v) —C(O)—R′, or

(vi) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl,

### W is

(i) —O—(C₁-C₆)alkyl, or

(ii) (C₁-C₆)-alkylene-C(O)OR, wherein
(ii.a) at least one of the carbon atoms of the (C₁-C₆)-alkylene is substituted with R₁ and/or R₂; and
(ii.b) at least one of the carbon atoms of the (C₁-C₆)-alkylene is replaced with an oxygen atom;
   R₁ and R₂ are each independently or simultaneously
(i) H,
(ii) (C₁-C₈)-alkyl,
(iii) (C₃-C₈)-alkyl, or
(iv) R₁ and R₂ together form a (C₃-C₈)-alkyl ring,
   R is H or (C₁-C₈)-alkyl, and
   n is 3, 4, 5, 6, 7, 8, 9, or 10,
   or a pharmaceutically acceptable salt, solvate, prodrug and/or stereoisomer thereof,
   and wherein the following compounds are excluded from the Formula (I)

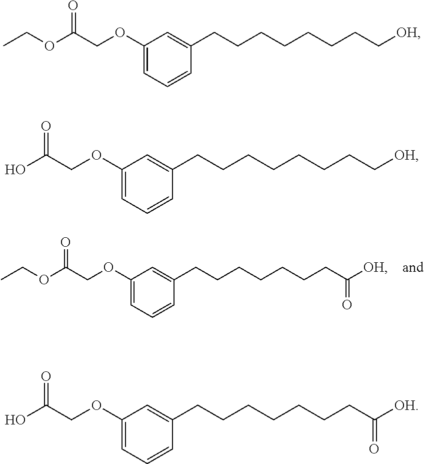 [SMILES: CCOC(=O)COc1cccc(CCCCCCCCO)c1 ; O=C(O)COc1cccc(CCCCCCCCO)c1 ; CCOC(=O)COc1cccc(CCCCCCCC(=O)O)c1 ; O=C(O)CCCCCCCc1cccc(OCC(=O)O)c1]

In one embodiment, Ring A is optionally substituted (C₆)-aryl or optionally substituted (C₅-C₆)-heteroaryl. In another embodiment, Ring A is optionally substituted (C₆)-aryl. In another embodiment of the disclosure, Ring A has the following structure

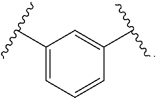 [SMILES: *c1cccc(*)c1]

In one embodiment, Ring A is optionally substituted once with OH, O—(C₁-C₆)-alkyl, —C(O)OH, —OC(O)—(C₁-C₆)-alkyl or —C(O)O—(C₁-C₆)-alkyl.

In another embodiment, Ring A has the structure

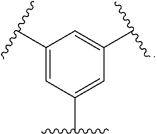 [SMILES: *c1cc(*)cc(*)c1]

In another embodiment of the disclosure, X is R′, —OR′, —C(O)O—R′, —C(O)—R′, or optionally substituted (C₆-C₁₀)-aryl, wherein R′ and R″ are each independently or simultaneously H, (C₁-C₈)-alkyl or (C₆-C₁₀)-aryl. In one embodiment, X is OR′ or —C(O)O—R′, wherein R′ and R″ are each independently or simultaneously H or (C₁-C₈)-alkyl. In a further embodiment, X is OH, —C(O)OH or —C(O)O—(C₁-C₈)-alkyl. In an embodiment, X is OH, —C(O)OH or —C(O)OCH₂CH₃.

In one embodiment of the disclosure, W is —O—CH₃, —C(R₁)(R₂)—O— or —O—C(R₁)(R₂)—, wherein R₁ and R₂ are each independently or simultaneously H, (C₁-C₆)-alkyl or (C₃-C₆)-alkyl, or R₁ and R₂ together form a (C₃-C₆)-cycloalkyl ring. In a further embodiment, W is —O—CH₃, —C(R₁)(R₂)—O— or —O—C(R₁)(R₂)—, wherein R₁ and R₂ are each independently or simultaneously (C₁-C₃)-alkyl or (C₃-C₆)-alkyl. In another embodiment, W is —OCH₃, —C(CH₃)(CH₃)—O— or —O—C(CH₃)(CH₃)—. In another embodiment, W is —OCH₃,

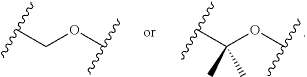

In an embodiment, R is H or (C₁-C₆)-alkyl. In one embodiment, H, CH₃ or CH₂CH₃.

In another embodiment of the disclosure, n is an integer which is 6, 7, 8, 9 or 10. In one embodiment, n is 7 or 8.

In another embodiment of the disclosure, the compound of the Formula (I) is

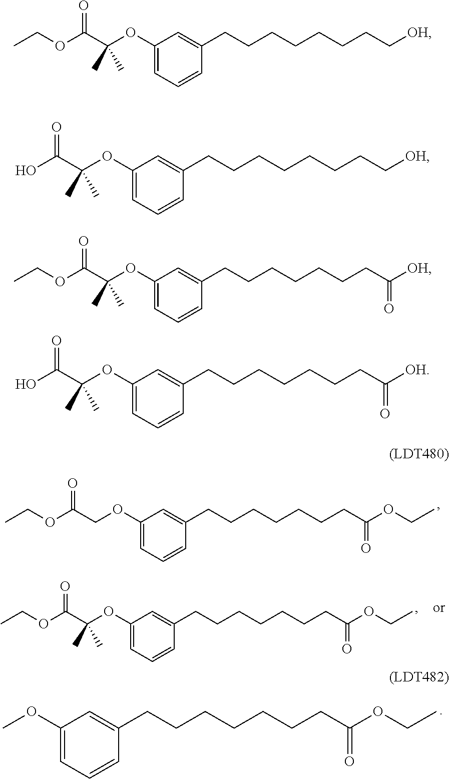

In another embodiment, the compounds of the Formula (I) are compounds of the Formula (Ia)

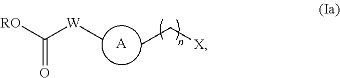

wherein

### Ring A is

(i) optionally substituted (C₆-C₁₀)-aryl, or

(ii) optionally substituted (C₅-C₁₀)-heteroaryl,

in which the optional substituents are selected from one to four of halo, OH, O—(C₁-C₆)-alkyl, (C₁-C₆)-alkyl, —C(O)OH, or —C(O)O—(C₁-C₆)-alkyl;

### X is

(i) R′,

(ii) —OR′,

(iii) —NR′R″,

(iv) —C(O)O—R′,

(v) —C(O)—R′, or

(vi) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl, W is (C₁-C₆)-alkylene, wherein at least two of the carbon atoms of the (C₁-C₆)-alkylene moiety are independently
(ii.a) substituted with R₁ and/or R₂; and
(ii.b) replaced with an oxygen atom; R₁ and R₂ are each independently or simultaneously

(i) (C₁-C₈)-alkyl,

(ii) (C₃-C₈)-alkyl, or

(iii) R₁ and R₂ together form a (C₃-C₈)-cycloalkyl ring,

R is H or (C₁-C₈)-alkyl, and
n is 3, 4, 5, 6, 7, 8, 9, or 10,
or a pharmaceutically acceptable salt, solvate, prodrug and/or stereoisomer thereof.

In one embodiment, the compound of the Formula (Ia) is

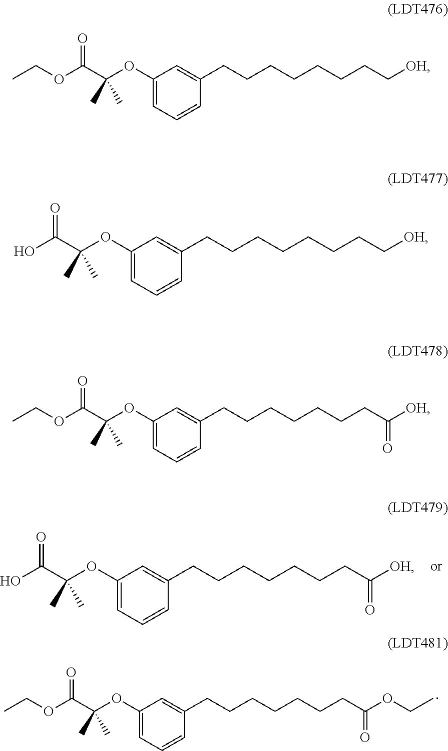

In one embodiment of the disclosure, the compound of the Formula (Ia) has the structure

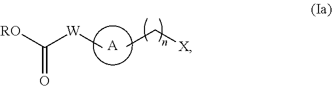

wherein

### Ring A is

(i) optionally substituted phenyl, or
(ii) optionally substituted (C₅-C₆)-heteroaryl, in which the optional substituents are selected from one to four of halo, OH, O—(C₁-C₆)-alkyl, (C₁-C₆)-alkyl, —C(O)OH, —OC(O)—(C₁-C₆)-alkyl or —C(O)O—(C₁-C₆)-alkyl;

### X is

(i) R′,

(ii) —OR′,

(iii) —C(O)O—R′,

(iv) —C(O)—R′, or

(v) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl,

W is (C₂-C₆)-alkylene, wherein
(ii.a) at least one carbon atom of the (C₂-C₆)-alkylene group is substituted with R₁ and/or R₂; and
(ii.b) at least one carbon atom of the (C₂-C₆)-alkylene group is replaced with an oxygen atom;

### R₁ is

(i) H,

(ii) (C₁-C₈)-alkyl, or

(iii) (C₃-C₈)-alkyl,

### R₂ is

(i) (C1-C₈)-alkyl, or

(ii) (C₃-C₈)-alkyl, or

R₁ and R₂ taken together form a (C₃-C₈)-cycloalkyl ring,
R is H or (C₁-C₈)-alkyl, and
n is 6, 7, 8, 9, or 10,
or a pharmaceutically acceptable salt, solvate, prodrug and/or stereoisomer thereof.

In one embodiment, in the compound of Formula (Ia), Ring A is optionally substituted phenyl. In another embodiment, Ring A is substituted once with OH, O—(C₁-C₆)-alkyl, —C(O)OH, —OC(O)—(C₁-C₆)-alkyl or —C(O)O—(C₁-C₆)-alkyl. In one embodiment, Ring A has the following structure

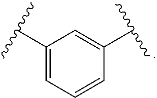 [SMILES: *c1cccc(*)c1]

In another embodiment, in the compound of Formula (Ia), Ring A has the following structure

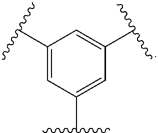 [SMILES: *c1cc(*)cc(*)c1]

In another embodiment, in the compound of Formula (Ia), X is R′, —OR′, —C(O)O—R′, —C(O)—R′, or optionally substituted (C₆-C₁₀)-aryl, wherein R′ and R″ are each independently or simultaneously H, (C₁-C₈)-alkyl or (C₆-C₁₀)-aryl. In one embodiment, X is OR′ or —C(O)O—R′, wherein R′ and R″ are each independently or simultaneously H or (C₁-C₈)-alkyl. In another embodiment, X is OH, —C(O)OH or —C(O)O—(C₁-C₈)-alkyl. In another embodiment X is OH, —C(O)OH or —C(O)O—(C₁-C₄)-alkyl. In another embodiment, X is OH, —C(O)OH or —C(O)OCH₂CH₃.

In another embodiment of the disclosure, in the compound of Formula (Ia), W is —C(R₁)(R₂)—O— or —O—C(R₁)(R₂)—, wherein R₁ is H, (C₁-C₆)-alkyl or (C₃-C₆)-cycloalkyl, R₂ is (C₁-C₆)-alkyl or (C₃-C₆)-cycloalkyl, or R₁ and R₂ taken together form a (C₃-C₆)-alkyl ring. In another embodiment, W is —C(R₁)(R₂)—O— or —O—C(R₁)(R₂)—, wherein R₁ is H, (C₁-C₃)-alkyl or (C₃-C₆)— cycloalkyl, and R₂ is (C₁-C₃)-alkyl or (C₃-C₆)-cycloalkyl. In one embodiment, W is —C(CH₃)(CH₃)—O— or —O—C(CH₃)(CH₃)—. In a further embodiment, W is

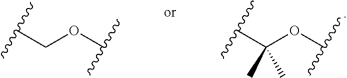

In one embodiment, in the compound of Formula (Ia), R is H or (C₁-C₆)-alkyl.

In one embodiment, R is H, CH₃ or CH₂CH₃.

In another embodiment, in the compound of Formula (Ia), n is 6, 7, 8, or 9, or optionally, n is 7 or 8.

In another embodiment of the disclosure, the compound of the Formula (Ia) is

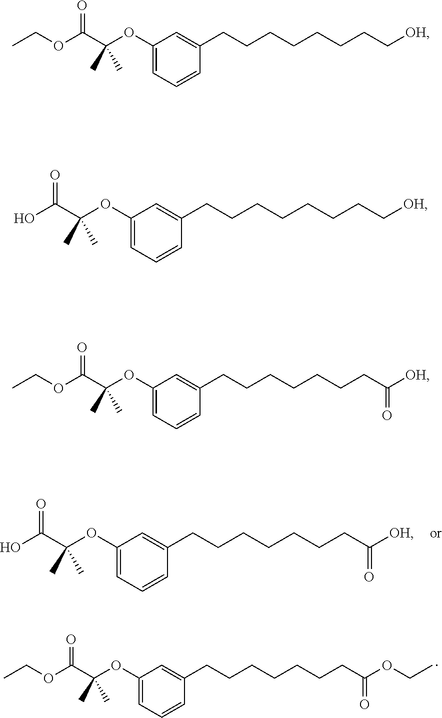

### Use of the Compounds of the Formula (II) and (III)

The present disclosure also includes a use of a compound of the Formula (II), as a PPAR modulator, and in particular, as a selective PPAR modulator, wherein the compound of the Formula (II) has the following structure

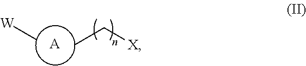

wherein

### Ring A is

(i) optionally substituted (C₆-C₁₀)-aryl, or

(ii) optionally substituted (C₅-C₁₀)-heteroaryl, in which the optional substituents are selected from one to four of halo, OH, O—(C₁-C₆)-alkyl, (C₁-C₆)-alkyl, —C(O)OH, or —C(O)O—(C₁-C₆)-alkyl; X is

(i) R′,

(ii) —OR′,

(iii) —NR′R″,

(iv) —C(O)O—R′,

(v) —C(O)—R′, or

(vi) —C(O)—NR′R″;

R′ and R″ are each independently or simultaneously

(i) H,

(ii) (C₁-C₈)-alkyl optionally substituted by (C₆-C₁₀)aryl, or

(iii) (C₆-C₁₀)-aryl optionally substituted by (C₁-C₈)alkyl or (C₆-C₁₀)aryl,

### W is

(i) —O—(C₁-C₆)alkyl, or

(ii) (C₂-C₆)-alkylene-C(O)OR, wherein
(ii.a) at least one carbon atom of the (C₂-C₆)-alkylene group is substituted with R₁ and/or R₂; and
(ii.b) at least one carbon atom of the (C₂-C₆)-alkylene group is replaced with an oxygen atom;
   R₁ and R₂ are each independently or simultaneously

(i) H,

(ii) (C₁-C₈)-alkyl,

(iii) (C₃-C₈)-alkyl, or

(iv) R₁ and R₂ together form a (C₃-C₈)-cycloalkyl ring,

R is H or (C₁-C₈)-alkyl, and
n is 3, 4, 5, 6, 7, 8, 9, or 10,
or a pharmaceutically acceptable salt, solvate, prodrug and/or stereoisomer thereof.

In one embodiment, Ring A is optionally substituted (C₆)-aryl or optionally substituted (C₅-C₆)-heteroaryl. In another embodiment, Ring A is optionally substituted (C₆)-aryl. In a further embodiment, Ring A has the following structure

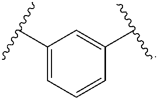 [SMILES: *c1cccc(*)c1]

In another embodiment of the disclosure, X is R′, —OR′, —C(O)O—R′, —C(O)—R′, or optionally substituted (C₆-C₁₀)-aryl, wherein R′ and R″ are each independently or simultaneously H, (C₁-C₈)-alkyl or (C₆-C₁₀)-aryl. In another embodiment, X is OR′ or —C(O)O—R′, wherein R′ and R″ are each independently or simultaneously H or (C₁-C₈)-alkyl. In one embodiment, X is OH, —C(O)OH or —C(O)O—(C₁-C₈)-alkyl. In another embodiment, X is OH, —C(O)OH or —C(O)OCH₂CH₃.

In another embodiment of the disclosure, W is —O—CH₃, —C(R₁)(R₂)—O— or —O—C(R₁)(R₂)—, wherein R₁ and R₂ are each independently or simultaneously H, (C₁-C₆)-alkyl or (C₃-C₆)-alkyl, or R₁ and R₂ together form a (C₃-C₆)-alkyl ring. In a further embodiment, W is —O—CH₃, —C(R₁)(R₂)—O— or —O—C(R₁)(R₂)—, wherein R₁ and R₂ are each independently or simultaneously (C₁-C₃)-alkyl or (C₃-C₆)-alkyl. In one embodiment, W is —OCH₃, —C(CH₃)(CH₃)—O— or —O—C(CH₃)(CH₃)—. In one embodiment, W is —OCH₃,

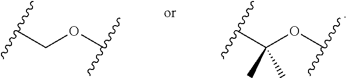

In one embodiment, R is H or (C₁-C₆)-alkyl. In another embodiment, R is H, CH₃ or CH₂CH₃.

In an embodiment, n is 6, 7, 8, 9 or 10. In one embodiment, n is 7 or 8.

In one embodiment of the disclosure, the compound of the Formula (II) is

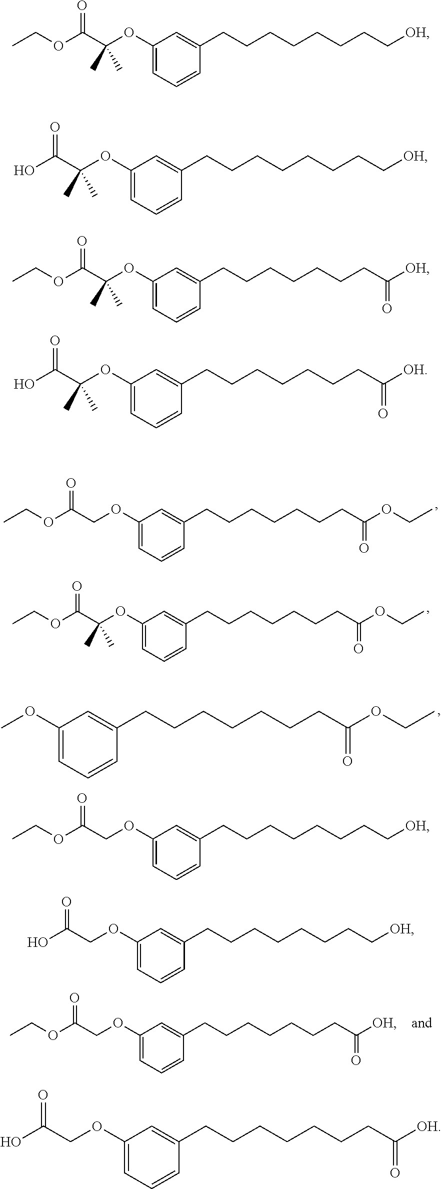

The present disclosure also includes a use of a compound of the Formula (III) as a selective PPAR modulator, wherein the compound of the Formula (III) has the following structure

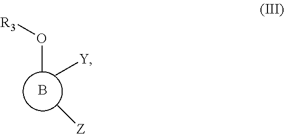

wherein

### Ring B is

(i) optionally substituted (C₆-C₁₀)-aryl, or

(ii) optionally substituted (C₅-C₁₀)-heteroaryl, in which the optional substituents are selected from one to four of OH, O—(C₁-C₆)-alkyl, (C₁-C₆)-alkyl, —C(O)OH, or —C(O)O—(C₁-C₆)-alkyl; R₃ is

(i) H,

(ii) (C₁-C₆)-alkyl,

(iii) —C(═O)—(C₁-C₆)-alkyl,

(iv) —(C₁-C₆)-alkylene-C(═O)—O—R₄, wherein the (C₁-C₆)-alkylene moiety is optionally substituted by one or more of (C₁-C₆)-alkyl; R₄ is

(i) H, or

(ii) (C₁-C₆)-alkyl

### Y is

(i) H, or

(ii) —C(O)OH;

Z is (C₁₀-C₂₀)alkyl, having 0-3 unsaturated bonds, or a pharmaceutically acceptable salt, solvate, prodrug and/or stereoisomer thereof.

In one embodiment, Ring B is optionally substituted (C₆)-aryl or optionally substituted (C₅-C₆)-heteroaryl. In another embodiment, Ring B is optionally substituted (C₆)-aryl. In another embodiment, Ring B has the following structure:

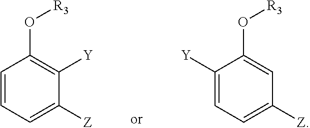

In another embodiment of the disclosure, R₃ is H, (C₁-C₃)-alkyl, —C(═O)—(C₁-C₃)-alkyl, or —(C₁-C₃)-alkylene-C(═O)—O—R₄, wherein the (C₁-C₃)-alkylene moiety is optionally substituted by one or more of (C₁-C₃)-alkyl. In a further embodiment, R₃ is H, CH₃, —C(O)CH₃, or —CH₂C(═O)O—R₄ optionally substituted by one or more methyl groups. In one embodiment, R₃ is

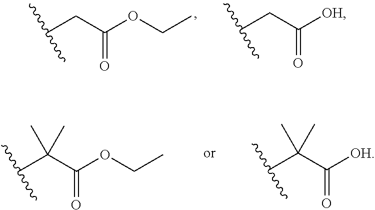

In another embodiment of the disclosure, Z is (C₁₂-C₁₈)alkyl, having 0-3 unsaturated bonds. In another embodiment, Z is (C₁₅)-alkyl, having 0-3 unsaturated bonds. In one embodiment, Z is

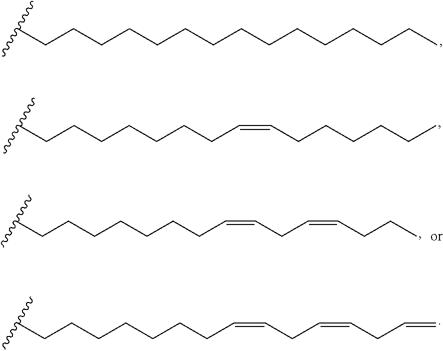 [SMILES: *CCCCCCCCCCCCCCC ; *CCCCCCC/C=C\CCCCCC ; *CCCCCCC/C=C\C/C=C\CCC ; *CCCCCCC/C=C\C/C=C\CC=C]

In one embodiment, the compound of the Formula (III) is

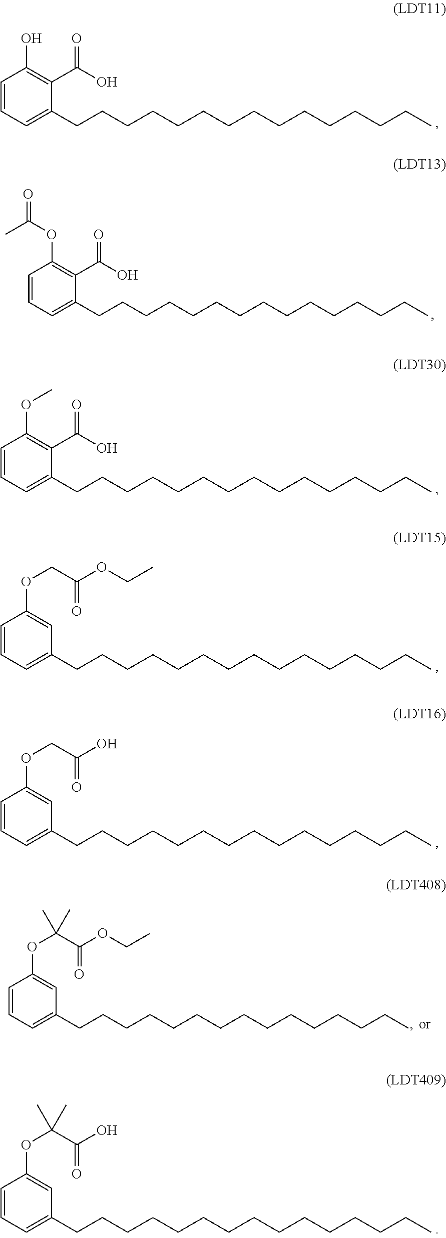

### Compositions

The present disclosure also includes pharmaceutical compositions comprising a compound of the Formula (I), (II) and/or (Ill) as defined above, or pharmaceutically acceptable salts, solvates, and prodrugs thereof, and a pharmaceutically acceptable carrier or diluent. The compounds are suitably formulated into pharmaceutical compositions for administration to subjects, preferably humans in a biologically compatible form suitable for administration in vivo.

The compositions containing the compounds of Formula (I), (II) and/or (III) can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (2003-20th edition) and in The United States Pharmacopeia: The National Formulary (USP 24 NF19) published in 1999. On this basis, the compositions include, albeit not exclusively, solutions of the substances in association with one or more pharmaceutically acceptable vehicles or diluents, and contained in buffered solutions with a suitable pH and iso-osmotic with the physiological fluids.

The compounds of Formula (I), (II) and/or (III) may be used pharmaceutically in the form of the free base, in the form of salts, solvates and as hydrates. All forms are within the scope of the disclosure. Acid and basic addition salts may be formed with the compounds of the disclosure for use as sources of the free base form even if the particular salt per se is desired only as an intermediate product as, for example, when the salt is formed only for the purposes of purification and identification. All salts that can be formed with the compounds of the disclosure are therefore within the scope of the present disclosure.

In another embodiment of the disclosure, the compounds of the Formula (I), (II) and/or (III) may be combined with other active agents such as active agents involved in the treatment of metabolic syndrome. For example, the compounds of the Formula (I), (II) and/or (III) may be combined with anti-diabetic medications such as metformin, or anti-atherosclerosis medications such as a statin.

### Methods of Medical Treatment

In one embodiment of the disclosure, the compounds of the Formula (I), (II) and (III) are PPAR modulators, and in particular, selective PPAR modulators for PPARα and PPARγ. In one embodiment, the compounds of the disclosure act as dual agonists for these receptors. In one embodiment of the disclosure, the compounds exhibit increased EC₅₀ values for PPARα and PPARγ relative to positive controls (for example, GW7647 and rosiglitazone, respectively). In another embodiment of the disclosure, the compounds of the Formula (I), (II) and/or (III) are partial agonists of PPARα while retaining full agonist activity of PPARγ. In an embodiment of the disclosure, selective PPAR modulators have an improved benefit to risk ratio when compared to full PPAR agonists. In one embodiment, as the compounds of the Formula (I), (II) and/or (III) are not full agonists of PPARα, they may have reduced side effects compared with other PPAR agonists.

In one embodiment of the disclosure, there is included a method of treating or preventing a disease or condition for which PPAR modulation provides a therapeutic benefit, comprising administering an effective amount of a compound of the Formula (I), (II) and/or (III) to a patient in need thereof. In one embodiment, the PPAR modulation is PPARα and PPARγ modulation. In another embodiment of the disclosure, the PPAR modulation using the compounds of the Formula (I), (II) and/or (III) comprises partial agonist activity against PPARα and full agonist activity against PPARγ.

In another embodiment of the disclosure, there is included a use of a compound of the Formula (I), (II) and/or (III), for treating or preventing a disease or condition for which PPAR modulation provides a therapeutic benefit.

In another embodiment, the disease or condition for which PPAR modulation provides a therapeutic benefit is metabolic syndrome. In one embodiment, metabolic syndrome includes obesity, hyperlipidemia, elevated fasting blood glucose, elevated blood pressure and/or low HDL cholesterol.

In another embodiment, the disease or condition for which PPAR modulation provides a therapeutic benefit is type 2 diabetes or cardiovascular disease.

In another embodiment, the disease or condition for which PPAR modulation provides a therapeutic benefit is a neurodegenerative disease. In one embodiment, the neurodegenerative disease is Alzheimer's disease, amyotrophic lateral sclerosis (ALS), Parkinson's disease, Huntington's disease, and multiple sclerosis. In one embodiment, the disease is Alzheimer's disease.

In another embodiment, the disease or condition for which PPAR modulation provides a therapeutic benefit is malaria.

In another embodiment, the disease or condition for which PPAR modulation provides a therapeutic benefit is irritable bowel syndrome.

### EXAMPLES

The operation of the disclosure is illustrated by the following representative examples. As is apparent to those skilled in the art, many of the details of the examples may be changed while still practicing the disclosure described herein.

Experimental Protocols

Materials and Methods

All reagents and solvents were obtained from commercial suppliers Tedia CoR and Sigma-AldrichR. Before use, the reagents and solvents were pretreated when necessary. The solvents were removed under reduced pressure on a TecnalR TE-211 apparatus. The acetylation reaction of cardanol for obtaining dihydroxy derivative (LDT71) was performed in a household microwave BrastempR BMK38ABHNA JetDeFrost model with a capacity of 38 L and power 900 W. All reactions were monitored by thin layer chromatography (TLC) using commercial plates precoated Kieselgel 60 F254. Visualization was performed by UV fluorescence (λmax=254-366 nm). Chromatographic separations were performed on a silica gel column G60 (70-230 mesh) SILICYCLER. Yields refer to chromatographically and spectroscopically pure compounds. Compounds were named following IUPAC rules. All melting points were determined on a Quimis 340/23 apparatus and are uncorrected. The identity and purity of intermediates and final compounds were ascertained through 1H NMR, 13C NMR and IR spectra. 1H NMR spectra were determined in deutered chloroform or methanol containing ca 1% tetramethylsilane as an internal standard, using a Bruker DOX 300 at 300 MHz and a Bruker Avance DXR 500. 13C spectra were determined in the same spectrometers at 75 MHz and 125 MHz, respectively, employing the same solvents. Chemical shifts (δ) are reported in ppm to the nearest 0.01 ppm using solvent as the internal standard. Coupling constants (J values) are given in Hz. The areas of the signals were obtained by electronic integration and their multiplicities described as: singlet (s), doublet (d), double doublet (dd), triplet (t), quartet (q), multiplet (m), broad signal (bs). The infrared spectra (IR) were obtained by the spectrometer Perkin ElmerR Spectrum BX (Central Analytical UCB) using potassium bromide discs (KBr) or as a liquid film on sodium chloride (NaCl) plate. The values for absorptions are reported in wave numbers, using as a unit the reciprocal centimeters (cm-1).

### Example 1: Synthesis and Characterization of Compounds

### 1.1 3-(8-Hydroxyoctyl)phenol (LDT71)

An erlenmeyer containing a solution of 12.00 g of mixture of cardanols (monoene, diene and triene) (39.406 mmol) distilled acetic anhydride (12.0 mL) and phosphoric acid (12 drops) was placed inside an unmodified household microwave oven and irradiated for 3 minutes (3×1′) at a power of 400 W. After, the residue was extracted with ethyl acetate (3×15.0 mL) and the combined organic fractions washed with solution of 5% sodium bicarbonate (20.0 mL), 10% hydrochloric acid solution (20.0 mL), brine (20.0 mL), and dried over anhydrous sodium sulfate. After evaporation of the solvent at reduced pressure, the reaction mixture was purified by chromatography on a silica gel (dichloromethane) affording the desired compound in 73% yield. Then, 10.00 g of the mixture of acetylated cardanols was diluted with dichloromethane (20.0 mL) and methanol (20.0 mL) in a ozonolysis flask of 250.0 mL. The flask was adapted to the ozonator with a stream of ozone for one 1.5 hour, in bath of dry ice/acetone. Next, the secondary ozonide was reduced with 5.900 g of sodium borohydride (158.704 mmol) in 60.0 mL of methanol. At the end of addition of sodium borohydride, the reaction remained for six hours under stirring. Then, the mixture was acidified with concentrated hydrochloric acid to pH 3, and it was extracted with ethyl acetate (3×30.0 mL). The combined organic fractions were washed with brine (30.0 mL) and dried over sodium sulfate. After evaporation of the solvent, the product was chromatographed on silica gel (dichloromethane and chloroform and then chloroform and ethanol) leading to hydroxylated derivative (LDT71) in 79% of yield: 79%. IR (KBr) vmax cm⁻¹: 3351, 2929, 2855, 1589, 1456. ¹H NMR (300 MHz, CDCl3): δ 1.30 (bs, 8H, CH2), 1.53-1.59 (m, 4H), 2.54 (t, J=7.6 Hz, 2H), 3.66 (t, J=6.6 Hz, 2H), 6.65 (dd, J=8.1 Hz, J=2.5 Hz, 1H), 6.67 (sl, 1H), 6.71 (d, J=7.6 Hz, 1H), 7.19 (t, J=7.8 Hz, 1H). RMN ¹³C (75 MHz, CDCl3): δ 25.8 (C3), 29.2 (C6), 29.4 (C5), 29.5 (C4), 31.3 (C7), 32.7 (C2), 35.9 (C8), 63.2 (C1), 112.8 (CH2′), 115.6 (CH6′), 120.8 (CH4′), 129.5 (CH5′), 144.9 (C3′), 156.0 (C—O1′).

### 1.2 8-(3-Methoxyphenyl)octan-1-01 (LDT72)

A mixture containing 1.00 g (3.782 mmol) of LDT71, 1.04 g of potassium carbonate (7.565 mmol), 0.60 mL of methyl iodide (9.456 mmol) and acetone (25.0 mL) was reflux for 24 hours. after, the acetone was evaporated under reduced pressure, and the mixture extracted with ethyl acetate (3×10.0 mL). The combined organic fractions were washed with 10% hydrochloric acid solution (30.0 mL), saturated brine (30.0 mL), and dried over anhydrous sodium sulfate. The solvent was evaporated under reduced pressure and the residue chromatographed on a silica gel (hexane-dichloromethane 1:1), yielding the derivative LDT72 in 80%. IR (KBr) νₘₐₓ cm⁻¹: 3368, 2929, 1602, 1465, 1260,1051, 776, 695. ¹H NMR (300 MHz, CDCl₃): δ1.33 (m, 8H, CH₂), 1.54-1.58 (m, 2H, CH₂), 1.60-1.63 (m, 2H, CH₂), 2.59 (t, J=6.0 Hz, 2H), 3,64 (t, J=6.0 Hz, 2H), 3.81 (s, 3H, ArOCH₃), 6.72-6.79 (m, 3H), 7.18-7.21 (m, 1H). ¹³C NMR (75 MHz, CDCl₃): δ 25.9 (C3); 29.6 (C6), 29.4 (C4), 29.5 (C5), 31.5 (C7), 32.9 (C2), 36.2 (C8), 55.3 (ArOCH₃), 110.9 (CH2′), 114.4 (CH6′), 121,1 (CH4′), 129.3 (CH5′), 144.7 (C3′), 159.7 (C—O1′).

### 1.3 8-(3-Methoxyphenyl)octanoic acid (LDT80)

To a solution of 0.50 g of LDT72 (2.115 mmol) in acetone (20.0 mL), under ice/water bath, was added dropwise Jones reagent until a brown coloration of the mixture was maintained for five minutes, indicating the end of the reaction. The excess of Jones reagent was deactivated by adding isopropyl alcohol (1.0 mL) and the mixture extracted with chloroform (2×10.0 mL), the organic fractions were washed with brine (10.0 mL), and dried over anhydrous sodium sulfate. After evaporation of the solvent at reduced pressure, the residue was chromatographed on a silica gel (dichloromethane) and then, dichloromethane and chloroform, leading to target LTD80 derivative in 96% yield. (white solid m.p. 46-48° C.). IR (KBr) νmax cm-1: 2927, 1708, 1595, 1459, 1272,1038. ¹H NMR (300 MHz, CDCl3): 1.21-1.33 (m, 9H, ArOCH3; C4-C6), 1.60-1.62 (m, 4H), 2.30-2.36 (m, 2H), 2.57 (t, J=7.7 Hz, 2H), 6.71-6.73 (m, 2H), 6,76 (d, J=7.7 Hz, 1H), 7,16-7.20 (m, 1H). ¹³C NMR (75 MHz, CDCl3): δ 18.4 (C4), 22.6 (C3), 24.9 (C6), 29.2 (C5), 31.3 (C7), 34.1 (AC2), 36.2 (C1), 55.3 (ArOCH3), 111.0 (CH2′), 114.4 (CH6′), 121.0 (CH4′), 129.3 (CH5′), 144.6 (C3′), 159.7 (C—O1), 179.0 (COON-8).

### 1.4 Ethyl 8-(3-methoxyphenyl)octanoate (LDT482)

A mixture of 0.10 g of LDT80 (0.399 mmol), 0.11 g of potassium carbonate (0.798 mmol) in acetone (7.0 mL) was stirred for 20 minutes, and then, 0.10 mL of ethyl iodide (1.198 mmol) were added. The mixture was refluxed for 2 hours. After, the solvent was concentrated under reduced pressure and the residue was extracted with ethyl acetate (3×10.0 mL). The combined organic phases were washed with a 10% hydrochloric acid solution (20.0 mL), brine (10.0 mL) and dried with anhydrous sodium sulfate. The solvent was evaporated under reduced pressure and the product was chromatographed on a silica gel (dichloromethane, chloroform and then chloroform and ethanol) to afford the compound LDT482 in 90% yield as an incolor liquid. IR (KBr) vmax cm-1: 2931, 1735, 1599, 1463, 1261, 1166. 1H NMR (300 MHz, CDCl₃): δ 1.26 (t, J=7.1 Hz, 3H), 1.28-1.34 (m, 6H, CH2), 1.63-1.66 (m, 4H, CH2), 2.29 (t, J=7.5 Hz, 2H), 2.58 (t, J=7.7 Hz, 2H), 3.80 (s, 3H, ArOCH3); 4.13 (q, J=7.1 Hz, 2H), 6.73 (dd, J=10.5 Hz, J=2.3 Hz, 1 H), 6,75 (sI, 1H); 6,79 (d, J=7.6 Hz, 1H), 7.17 (t, J=7.8 Hz, 1H). 13C NMR (75 MHz, CDCl3): δ 14.4 (RCO2CH2CH3), 25.0 (C3), 25.1 (C4), 29.2 (C5), 29.3 (C6), 31.5 (C7), 34.5 (C2); 36.2 (C8), 55.3 (ArOCH3), 60.3 (Ar/CO2CH2CH3), 111.0 (CH2′), 114,4 (CH6′), 121.0 (C4′), 129.3 (CH5′), 144.6 (C3′), 159.8 (C—O1′), 174.0 (Ar/CO2CH2CH3).

### 1.5 Ethyl 2-(3-(8-hydroxyoctyl)phenoxy)acetate (LDT296)

A mixture containing 1.00 g of LDT71 (4.497 mmol), 1.240 g of potassium carbonate (8.995 mmol) and acetone (50.0 mL) was stirred for 20 minutes, and then, 0.62 mL of ethyl 2-bromoacetate (5.622 mmol) were added. The reaction was stirred for 24 hours at room temperature, and then, extracted with ethyl acetate (2×30.0 mL). The combined organic fractions were washed with a 10% hydrochloric acid solution (30.0 mL), brine (20.0 mL), and dried over anhydrous sodium sulfate. The solvent was evaporated under reduced pressure and the residue chromatographed on a silica gel column (dichloromethane:chloroform 2:1), yielding the ester derivative LDT 296 in 62% yield as a in color liquid. IR (film) vmax cm-1: 3421, 2929, 2955, 1761, 1596, 1458, 1205, 1093. ¹H NMR (300 MHz, CDCl₃): δ 1.27-1.32 (m, 3H, ArOCH2CO2CH2CH3; 8H, CH2, C3-C6), 1.53-1.59 (m, 4H, CH2), 2.57 (t, J=7.5 Hz, 2H), 3.63 (t, J=6.6 Hz, 2H), 4.27 (q, J=7.1 Hz, 2H, ArOCH2CO2CH2CH3), 4.61 (s, 2H, ArOCH2CO2CH3), 6.71 (dd, J=8.1 Hz, J=2.5 Hz, 1H), 6.76 (bs, 1H), 6.81 (d, J=7.6 Hz, 1H), 7.19 (t, J=7.8 Hz, 1H). ¹³C NMR (75 MHz, CDCl₃): δ 14.3 (ArOCH2CO2CH2CH3), 25.9 (C3), 29.3 (C6), 29.5 (C5), 29.6 (C4), 31.4 (C7), 33.0 (C2), 36.1 (C8); 61.5 (ArOCH2CO2CH2CH3), 63.2 (CH₂OH-1), 65.7 (ArOCH2CO2CH2CH3), 111.6 (CH2′), 115.3 (CH4′), 122.1 (CH4′), 129.4 (CH5′), 144.9 (C3′), 158.1 (C—O1′), 169.3 (ArOCH2CO2CH2CH3).

### 1.6 8-(3-(2-Ethoxy-2-oxoethoxy)phenyl)octanoic acid (LDT298)

To a solution of 0.460 g of LDT296 (1.491 mmol) in acetone (20.0 mL), under ice/water bath, Jones reagent was added dropwise until a brown coloration of the reaction system was maintained for five minutes, indicating the end of the reaction. The excess of Jones reagent was deactivated by adding isopropyl alcohol (1.0 mL) and the mixture was extracted with chloroform (2×15.0 mL). The combined fractions were washed with saturated saline solution (10.0 mL), and dried anhydrous sodium sulfate. After evaporation of the solvent at reduced pressure, the residue was chromatographed on a silica gel (chloroform, chloroform and ethanol) leading to target LTD298 derivative in 83% yield. IR (film) vmax cm-1: 2930, 2857, 1760, 1735, 1603, 1457, 1204, 1093. ¹H NMR (300 MHz, CDCl₃): δ 1.27-1.33 (m, 9H, ArOCH2CO2CH2CH3, C4-C6), 1.62-1.60 (m, 4H, CH2), 2.34 (t, J=7.5 Hz, 2H), 2.57 (t, J=7.6 Hz, 2H), 4.28 (q, J=7.1 Hz, 2H, ArOCH2CO2CH2CH3), 4.61 (s, 2H, ArOCH2CO2CH3), 6.72 (dd, J=8.1 Hz, J=2.0 Hz, 1H), 6.75 (bs, 1H), 6.77 (dl, J=7.5 Hz, 1H), 7.19 (t, J=7.8 Hz, 1H). ¹³C NMR (75 MHz, CDCl₃): δ 14.3 (ArOCH2CO2CH2CH3), 24.8 (C3), 29.1 (C5), 29.2 (C4), 29.2 (C6), 31.3 (C7), 34.2 (C2), 36.0 (C8), 61.5 (ArOCH2CO2CH2CH3), 65.6 (ArOCH2CO2CH2CH3), 111.7 (CH2′), 115.3 (CH6′); 122.1 (CH4′), 129.4 (CH5′), 144.8 (C3′), 158.0 (C—O1′), 169.3 (ArOCH2CO2CH2CH3), 179.9 (C1).

### 1.7 Ethyl 8-(3-(2-ethoxy-2-oxoethoxy)phenyl)octanoate (LDT480)

A mixture containing 0.10 g of LDT298 (0.310 mmol), 0.080 g of potassium carbonate (0.620 mmol), and acetone (10.0 mL) was stirred for 20 minutes, and then, 0.04 mL of ethyl iodide (0.620 mmol) were added. The mixture was refluxed for 2 hours. After, the solvent was concentrated and the mixture was extracted with ethyl acetate (3×10.0 mL). The combined organic phases were washed with a 10% hydrochloric acid solution (20.0 mL), brine (10.0 mL) and dried with anhydrous sodium sulfate. The solvent was evaporated under reduced pressure and the residue was chromatographed on a silica gel (dichloromethane, chloroform and then, chloroform and ethanol), affording the compound LDT480 in 98%. IR (KBr) vmax cm⁻¹: 2931, 2856, 1762, 1735, 1603, 1586, 1486, 1448, 1201, 1094. ¹H NMR (300 MHz, CDCl₃): δ 1.22-1.31 (m, 12H, ArOCH2CO2CH2CH3, Ar/CO2CH2CH3, C4-C6), 1.58-1.63 (m, 4H), 2.27 (t, J=7.5 Hz, 2H), 2.56 (t, J=7.7 Hz, 2H), 4.11 (q, J=7.1 Hz, 2H, ArOCH2CO2CH2CH3), 4.26 (q, J=7.1 Hz, 2H, Ar/CO2CH2CH3), 4.60 (s, 2H, ArOCH2CO2CH3), 6.69 (dd, J=6.5 Hz, J=2.3 Hz, 1H), 6.74 (sl, 1H), 6.80 (dl, J=7.6 Hz, 1H), 7.17 (t, J=7.8 Hz, 1H). RMN ¹³C (75 MHz, CDCl3): δ 14,3 (ArOCH2CO2CH2CH3); 14,4 (Ar/CO2CH2CH3); 25,1 (Ar/CH2-3); 29,2 (Ar/CH2-5); 29,2 (Ar/CH2-4); 29,2 (Ar/CH2-6); 31,3 (Ar/CH2-7); 34,5 (Ar/CH2-2); 36,0 (Ar/CH2-8); 60,3 (Ar/CO2CH2CH3); 61,4 (ArOCH2CO2CH2CH3); 65,6 (ArOCH2CO2CH2CH3); 111,6 (Ar-2′-CH); 115,2 (Ar-4′-CH); 122,1 (Ar-6′-C); 129,4 (Ar-5′-C); 144,8 (Ar— 3′-C); 158,0 (Ar-1′-C—O); 169,2 (ArOCH2CO2CH2CH3); 174,0 (Ar/CO2CH2CH3).

### 1.8 Ethyl 2-(3-(8-hydroxyoctyl)phenoxy)-2-methylpropanoate (LDT476)

A mixture containing 1.50 g of potassium carbonate (10.794 mmol), 1.70 g of potassium iodide (10.794 mmol), 1.60 mL of ethyl α-bromoisobutyrate (10.794 mmol) and acetonitrile (20.0 mL) was stirred for 20 minutes, and then, were added 1.20 g of LDT71 (5.397 mmol). The reaction was refluxed for 48 hours. After, the solvent was concentrated and the mixture was extracted with ether (2×30.0 mL). The combined organic fractions were washed with 10% hydrochloric acid solution (30.0 mL), brine (30.0 mL) and dried over sodium sulfate. The solvent was evaporated under reduced pressure and the residue chromatographed on a silica (dichloromethane), to afford the derivative LDT476 in 65% yield. IR (film) vmax cm⁻¹: 3421, 2929, 2855, 1734, 1602, 1583, 1485, 1466, 1179, 1023. ¹H NMR (300 MHz, CDCl₃): δ 1.21-1.26 (m, 4H, ArOC(CH3)2CO2CH2CH3; 9H, CH2, C2-C6), 1,42-1,52 (m, 11H, ArOC(CH3)2CO2CH2CH3, C7), 2.53 (t, J=7.5 Hz, 2H), 3.61 (t, J=6.6 Hz, 2H), 4.22 (q, J=7.1 Hz, 2H, ArOC(CH3)2CO2CH2CH3), 6.62 (dd, J=8.1 Hz, J=2.4 Hz, 1H), 6.67 (sI, 1H), 6.79 (d, J=7.5 Hz, 1H), 7.10 (t, J=7.8 Hz, 1H). ¹³C NMR (75 MHz, CDCl3): δ 14.2 (ArOC(CH3)2CO2CH2CH3), 25.5 (ArOC(CH3)2CO2CH2CH3), 25.8 (C3), 29.2 (C6), 29.4 (C5), 29.5 (C4), 31.3 (C7), 32.9 (C2), 36.0 (C8), 61.5 (ArOC(CH3)2CO2CH2CH3), 63.0 (C1), 79.1 (ArOC(CH3)2CO2CH2CH3), 116.3 (CH2′), 119.5 (CH6′), 122.4 (C4′), 128.9 (CH5′), 144.3 (C3′), 155.5 (C—O1′), 174.6 (ArOC(CH3)2CO2CH2CH3).

### 1.9 2-(3-(8-hydroxyoctyl)phenoxy)-2-methylpropanoic acid (LDT477)

To a solution of 0.30 g of LDT476 (0.891 mmol) in tetrahydrofuran (7.0 mL) was added a solution of 0.08 g of lithium hydroxide (3.566 mmol) dissolved in distilled water (3.0 mL), and transfer catalyst phase AliquatR (3 drops). The mixture was refluxed for 4 hours. Then, the mixture was acidified with concentrated hydrochloric acid to pH 1 and it was extracted with ethyl acetate (3×10.0 mL). The combined organic fractions were washed with brine (10.0 mL) and dried with sulfate anhydrous sodium. The solvent was evaporated under reduced pressure and the residue was chromatographed on silica gel (dichloromethane, chloroform and then, chloroform and ethanol), affording the compound LDT477 in 99% yield. IR (KBr) vmax cm-1: 2928, 2856, 1719, 1603, 1485, 1466, 1152, 1010. 1H NMR (300 MHz, MeOD): δ 1.24-1.30 (m, 11H), 1,60-165 (m, 12H, ArOC(CH3)2CO2H), 2.54 (t, J=8.0 Hz, 2H), 3.60-3.66 (m, 2H), 6.69 (dd, J=8.0 Hz, J=2.3 Hz, 1H), 6.75 (sl, 1H), 6.85 (d, J=7.6 Hz, 1H), 7.15 (d, J=4.6 Hz, 1H). 13C NMR (75 MHz, MeOD): δ 25.4 (ArOC(CH3)2CO2H), 25.6 (C3), 28.6 (C6), 29.2 (C4), 29.3 (C5), 31.2 (C7), 32.5 (C2), 35.8 (C8), 63.1 (C1), 79.4 (ArOC(CH3)2CO2H), 117.4 (CH2′), 120.3 (CH6′), 123.2 (CH4′), 129.1 (CH5′), 144.4 (C3′), 154.9 (C—O1′), 177.6 (ArOC(CH3)2CO2H).

### 1.10 8-(3-((1-ethoxy-2-methyl-1-oxopropan-2-yl)oxy)phenil)octanoic acid (LDT478)

To a solution of 0.600 g of LDT476 (1.783 mmol) in acetone (20.0 mL), under ice/water bath, was added dropwise Jones reagent until the brown coloration of the reaction was maintained for five minutes, indicating the end of the reaction. The excess of Jones reagent was deactivated by adding isopropyl alcohol (2.0 mL) and the mixture extracted with chloroform (2×15.0 mL). The fractions combined were washed with 10% hydrochloric acid solution (30.0 mL), brine (10.0 mL), and dried over sodium sulfate. After evaporation of the solvent at reduced pressure, the residue was chromatographed on a silica gel (dichloromethane, chloroform and then, chloroform and ethanol), leading to target derivative LTD478 in 80% yield. IR (KBr) vmax cm-1: 2931, 2856, 1734, 1709, 1602, 1458,1142, 1024. 1H NMR (300 MHz, CDCl3): δ 1.25 (t, J=7.1 Hz, 3H), 1.32 (bs, 6H), 1.55-1.64 (bs, 11H, ArOC(CH3)2CO2CH2CH3), 2.34 (t, J=7.5 Hz, 2H), 2.53 (t, J=7.6 Hz, 2H), 4.23 (q, J=7.1 Hz, 2H), 6.63 (dd, J=6.0 Hz, J=1.9 Hz, 1H), 6.68 (sl, 1H), 6.80 (d, J=7.6 Hz, 1H), 7.12 (t, J=7.8 Hz, 1H). ¹³C NMR (75 MHz, CDCl3): δ 14.2 (ArOC(CH3)2CO2CH2CH3), 24.8 (ArOC(CH3)2CO2CH2CH3), 25.6 (C3), 29.1 (C4), 29.2 (C6), 31.3 (C7), 34.2 (C2), 36.0 (C8), 61.5 (ArOC(CH3)2CO2CH2CH3), 79.1 (ArOC(CH3)2CO2CH2CH3), 116.3 (CH2′), 119.5 (CH6′), 122.4 (CH4′), 128.9 (CH5′), 144.3 (C3′), 155.5 (C01′), 174.6 (ArOC(CH3)2CO2CH2CH3), 180,0 (C1).

### 1.11 8-(3-((2-carboxypropan-2-yl)oxy)phenyl)octanoic acid (LDT479)

To a solution of 0.26 g of LDT478 (0.821 mmol) in tetrahydrofuran (5.0 mL) was added a solution of 0.08 g of lithium hydroxide (3.287 mmol) dissolved in distilled water (3.0 mL), and transfer catalyst phase AliquatR (3 drops). The mixture was refluxed for 4 hours. Then, the mixture was acidified with concentrated hydrochloric acid to pH 1 and it was extracted with ethyl acetate (3×10.0 mL). The combined organic fractions were washed with brine (10.0 mL) and dried with sulfate anhydrous sodium. The solvent was evaporated under reduced pressure and the residue was chromatographed on silica gel (dichloromethane), affording the compound LDT479 in 82% yield. IR (KBr) vmax cm⁻¹: 2931, 2856, 1710, 1604, 1584, 1485, 1466, 1154,1009. ¹H NMR (300 MHz, MeOD): δ 1.23-1.28 (m, 6H), 1.59-1.61 (m, 10H, ArOC(CH3)2CO2H), 2.35 (t, J=7.3 Hz, 2H), 2.55 (t, J=7.6 Hz), 6.74 (d, J=7.2 Hz, 1H), 6.85 (s, 1H), 7.16 (d, J=7.6 Hz, 1H), 7.18 (t, J3=7.8 Hz, 1H). 13C NMR (75 MHz, MeOD): δ24.8 (ArOC(CH3)2CO2H), 25.3 (C3), 28.7 (C5), 28.9 (C4), 28.9 (C6), 31.2 (C7), 34.2 (C2), 35.9 (C8), 79.3 (ArOC(CH3)2CO2H), 117.6 (CH2′), 120.3 (CH6′), 123.3 (CH4′), 129.1 (CH5′), 144.5 (C3′), 154.9 (C—O1′), 179.5 (ArOC(CH3)2CO2H), 180.5 (C1).

### 1.12 Ethyl 8-(3-((1-ethoxy-2-methyl-1-oxopropan-2-yl)oxy)phenyl)octanoate (LDT481)

A mixture containing 0.17 g of LDT478 (0.485 mmol), 0.13 g of potassium carbonate (0.970 mmol), and acetone (10.0 mL) was stirred for 20 minutes, and then, 0.08 mL of ethyl iodide (0.970 mmol) were added. The mixture was refluxed for 2 hours. After, the solvent was concentrated and the mixture was extracted with ethyl acetate (3×10.0 mL). The combined organic phases were washed with a 10% hydrochloric acid solution (20.0 mL), brine (10.0 mL) and dried with anhydrous sodium sulfate. The solvent was evaporated under reduced pressure and the residue was chromatographed on a silica gel (dichloromethane, chloroform and then, chloroform and ethanol), affording the compound LDT481 in 79% yield. (white solid m.p. 39-42° C.). IR (KBr) ν max cm⁻¹: 2926, 2856, 1710, 1600, 1527, 1493, 1468, 1272, 1193. 1H NMR (300 MHz, CDCl3): δ 1.22-1.30 (m, 12H), 1,58 (bs, 11H), 2.27 (t, J=7.5 Hz, 2H), 2.53 (t, J=7.6 Hz, 2H), 4.11 (q, J=7.1 Hz, 2H), 4.22 (q, J=7.1 Hz, 2H), 6.63 (dd, J=6.1 Hz, J=2.0 Hz, 1H), 6.67 (sI, 1H), 6.79 (dl, J=7.6 Hz, 1H), 7,11 (t, J=7.8 Hz, 1H). 13C NMR (75 MHz, CDCl3): δ 14.2 (ArOC(CH3)2CO2CH2CH3), 14.4 (Ar/CO2CH2CH3), 25.1 (C3), 29.2 (C5), 29.2 (C4), 29.3 (C6), 31.4 (C7), 34.5 (C2), 36.0 (C8), 60.3 (Ar/CO2CH2CH3), 61.5 (ArOC(CH3)2CO2CH2CH3), 79.1 (ArOC(CH3)2CO2CH2CH3), 116.2 (CH2′), 119.5 (CH6′), 122.4 (CH4′), 128.9 (CH5′), 144.3 (C3′), 155.6 (C—O1′), 174.0 (ArOC(CH3)2CO2CH2CH3), 174.6 (Ar/CO2CH2CH3).

### Example 2: Biological Assays

2.1 Luciferase Assays

HEK293 cells were maintained in Dulbecco's modified Eagle's medium supplemented with 10% fetal bovine serum. Cell transfection was performed in media containing 10% charcoal-stripped fetal bovine serum using calcium phosphate in 96-well plates. The total amount of plasmid DNA (150 ng/well) included 50 ng UAS-Iuc reporter, 20 ng β-galactosidase, 15 ng nuclear receptor (GAL4-hPPARα and GAL4-hPPARγ) and pGEM filler plasmid. Ligands were added at 6 to 8 hours post transfection. Cells harvested 14 to 16 hours later were assayed for luciferase and β-galactosidase activity.

2.2 Primary Hepatocyte Assays

Mouse primary hepatocytes were isolated by collagenase perfusion as previously described (Patel et al. 2011). Cells were plated onto type I collagen-coated plates at 0.5×106 cells per well for 2 hours in attachment media (William's E Media, 10% charcoal stripped FBS, 1× penicillin/streptomycin, and 10 nM insulin), and then switched to overnight media (M199 Media, 5% charcoal stripped FBS, 1× penicillin/streptomycin, and 1 nM insulin). Ligand treatments were carried out on the following day in M199 media without FBS. Cells were harvested 24 hrs later for RNA extraction.

2.3 3T3-L1 Differentiation Assays

3T3-L1 cells were maintained in Dulbecco's modified Eagle's medium supplemented with 10% fetal calf serum. Adipocyte differentiation was induced in two-days postconfluent cells by treating the cells with 100 μg/ml isobutylmethylxanthine, 1 μM dexamethasone, and 5 μg/ml insulin with 10% FBS in DMEM (Day 0). Rosiglitazone and LDT ligands were added at the start of differentiation. Two days later, cells were switched to the medium containing 5 μg/ml insulin with 10% FBS. After an additional 72 hrs, cells were switched to maintenance media containing 10% FBS in DMEM. Thereafter, the media was changed every 2 days. Cells were harvested on day 11 for RNA expression and Oil Red O was used to estimate lipid accumulation.

2.4 Oil Red O Staining

Following 11 days of differentiation, cells were washed with PBS twice and fixed in 10% neutral buffered formalin (Sigma) for 1 h at room temperature, followed by two ddH₂O and two washes with 60% isopropanol. Cells were then stained with 0.6% (w/v) Oil Red O solution (60% isopropanol, 40% water) for 10 min at room temperature, followed by five washes with ddH2O to remove unbound dye and images were taken with Leica M205 FA microscope.

2.5 RNA Isolation, cDNA Synthesis and Real Time QPCR Analysis

Total RNA was extracted from cells using RNA STAT-60 (Tel-Test Inc.), followed by DNase I treatment (RNase-free; Roche), and reverse transcribed into cDNA with random hexamers using the High Capacity Reverse Transcription System (ABI; Applied Biosystems). Real-time quantitative PCR (QPCR) analysis was performed on an ABI 7900 in 384-well plates using 2×SYBR Green PCR Master Mix (ABI). Relative mRNA levels were calculated using the comparative Ct method normalized to cyclophilin mRNA.

### DISCUSSION

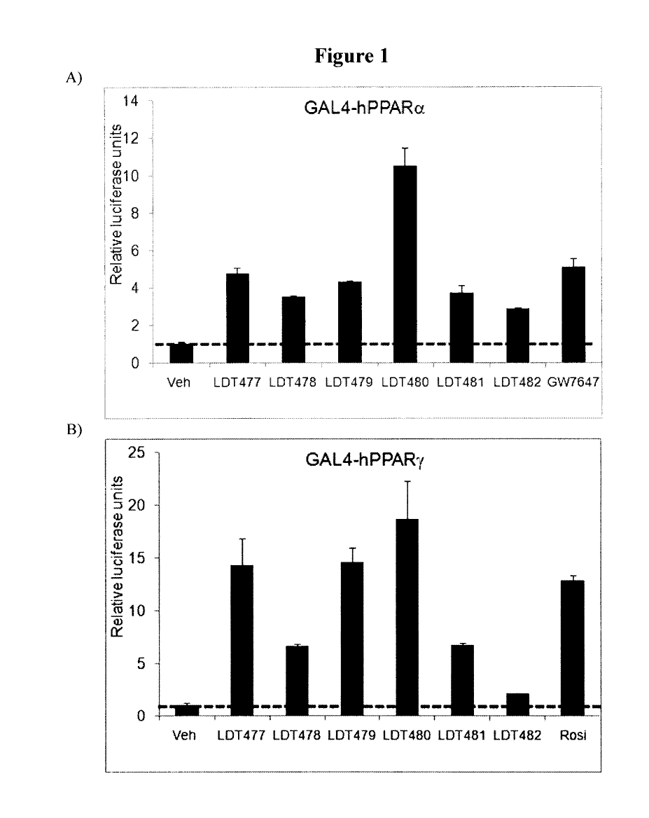

To identify PPARα and PPAR γ agonists, compounds of the Formula (I) (in particular compounds of the Formula (Ia)) were analyzed for activity using the GAL4-hPPARα/γ-UAS-luciferase reporter system in transiently transfected HEK293 cells as shown in FIG. 1. All of the compounds tested (dosed at 50 μM) showed activity on both PPARα (FIG. 1A) and PPARγ (FIG. 1B), with at least 3-fold increase in response when compared to Vehicle. LDT477 and LDT480 were the most active compounds in this assay.

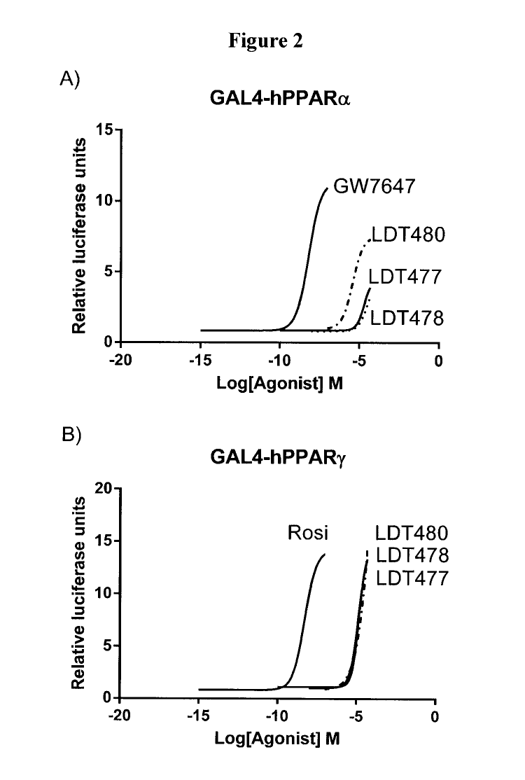

To determine the individual potencies of the tested compounds, dose-response analyses were carried out in the HEK293 cells using the previously described GAL4-hPPARα/γ-UAS-luciferase reporter system as shown in FIG. 2. LDT477, LDT478 and LDT480 were found to be less potent partial PPARα agonists when compared to the full agonist GW7647, with EC50 values of 20 μM, 37 μM and 3.9 μM, respectively (FIG. 2A). Interestingly, these compounds had a full agonist profile on the PPARγ receptor when compared to rosiglitazone control, with EC50 values of 14 μM, 14 μM, and 21 μM, respectively (FIG. 2B).

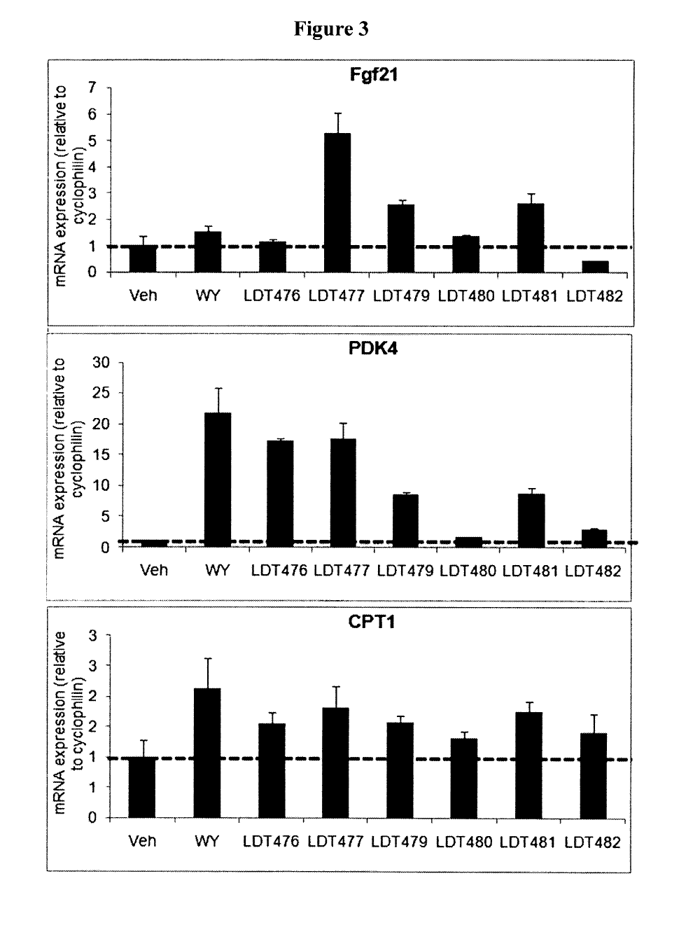

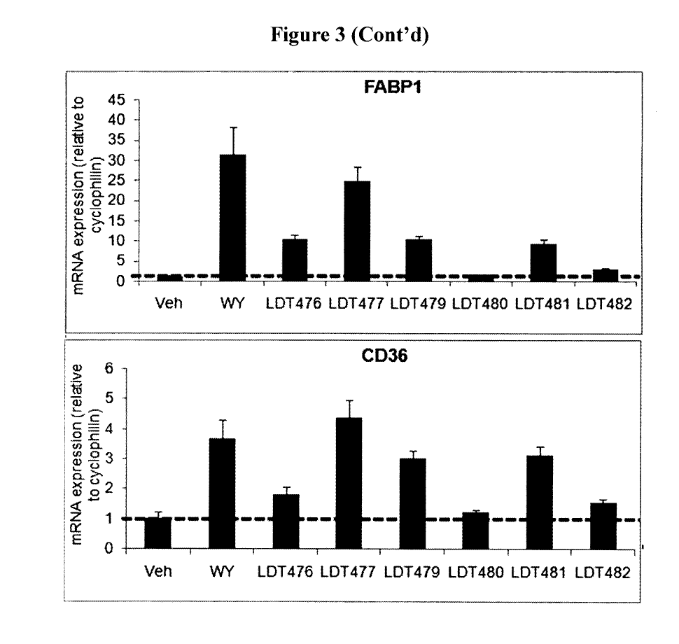

Classically, the activation of PPARα leads to a gene expression cascade that increases intracellular fatty acid uptake and β-oxidation in the liver. The ability of the compounds of the Formula (I) to upregulate the expression of PPARα target genes in primary mouse hepatocytes was examined. Treatment of primary hepatocytes with 50 μM of the compounds as shown in FIG. 3 resulted in the upregulation of Fgf21 expression, a hormone which has potent anti-diabetic properties by promoting fatty acid oxidation and ketone body production. The expression of CPT-1, a rate limiting enzyme in β-oxidation, was also increased following treatment. Moreover, the expression of PDK4, an enzyme which allows for preferential utilization of fatty acids as an energy source instead of glucose, was also upregulated. Finally, treatment with the compounds of the Formula (I) upregulated the expression of genes involved in fatty acid uptake, FABP1 and CD36. Overall, these results indicate that the compounds of the Formula (I) promote fatty acid oxidation in the liver. It is important to note, that despite the fact that the compounds exhibited partial agonist activity in the luciferase-reporter assays (FIG. 1) they showed similar efficacy as the positive control WY for PPARα target gene activation. In summary, the majority of the compounds were able to activate PPARα in primary hepatocytes to promote a gene expression program that facilitates fatty acid uptake and oxidation. These are the beneficial effects of PPARα-targeting drugs that are used to correct dyslipidemia in type 2 diabetes and metabolic disorders.

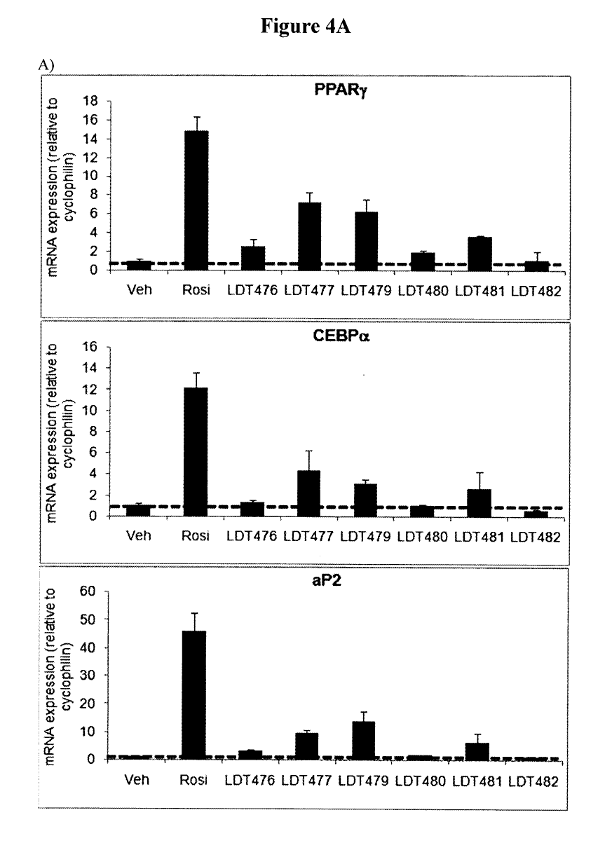

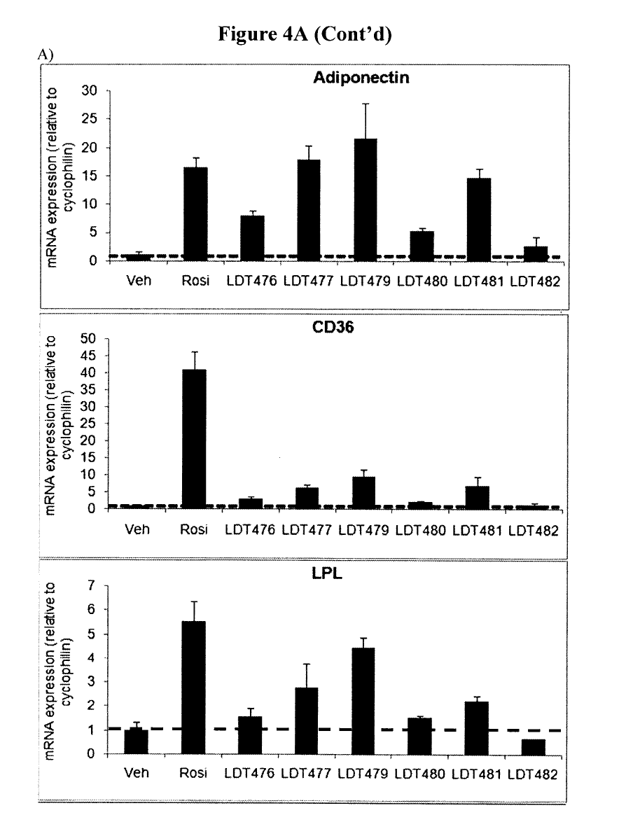

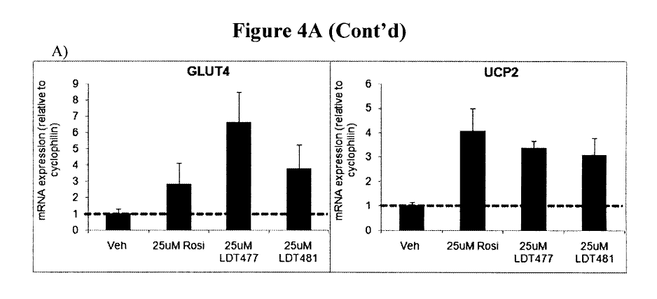

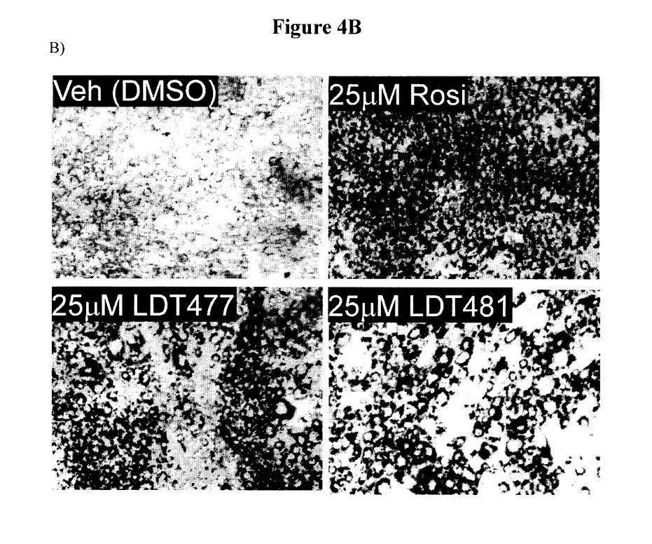

TZDs are well characterized inducers of adipocyte differentiation via PPARγ-mediated induction of the adipogenic program. The ability of the compounds of the Formula (I) to upregulate the expression of PPARγ target genes in a 3T3-L1 cell differentiation assay was examined. Treatment of 3T3-L1 pre-adipocytes with 25 μM of the compounds as shown in FIG. 4 resulted in the upregulation of PPARγ and CEBPα, two master regulators of adipocyte differentiation. The expression of adiponectin was also elevated following treatment with the compounds. Adiponectin is secreted by mature adipocytes and improves insulin sensitivity by stimulating AMPK, increasing glucose uptake in muscle, and decreasing gluconeogenesis in the liver. In addition, adiponectin also decreases adipose tissue inflammation. The expression of GLUT4, the predominant glucose uptake transporter in adipocytes, was also upregulated by LDT477 and LDT481. In type 2 diabetes, adipocytes are not able to uptake the glucose following insulin stimulation due to insulin resistance. Therefore, LDT477 and LDT481 may exert beneficial effects on insulin resistance by increasing the expression of GLUT4 in adipose tissue. TZDs have the further beneficial effect of promoting lipid oxidation in fat through coordinated uptake of fatty acids via induction of LPL, CD36 and AP2. Moreover, the expression of uncoupling protein 2 (UCP2) in adipose tissue, which is important for mediating energy expenditure, was also upregulated by LDT compounds. In line with the gene expression data, Oil red 0 staining (FIG. 4B) showed that LDT477 and LDT481 were able to induce adipocyte differentiation. Overall, certain compounds of the Formula (I) were found to possess the beneficial effects of PPARγ activation by showing potent increases in adiponectin levels with an overall lower adipocyte differentiation rate, which indicates the compounds can be therapeutically beneficial and permit insulin sensitization without the undesirable weight gain that occurs with TZD therapy.

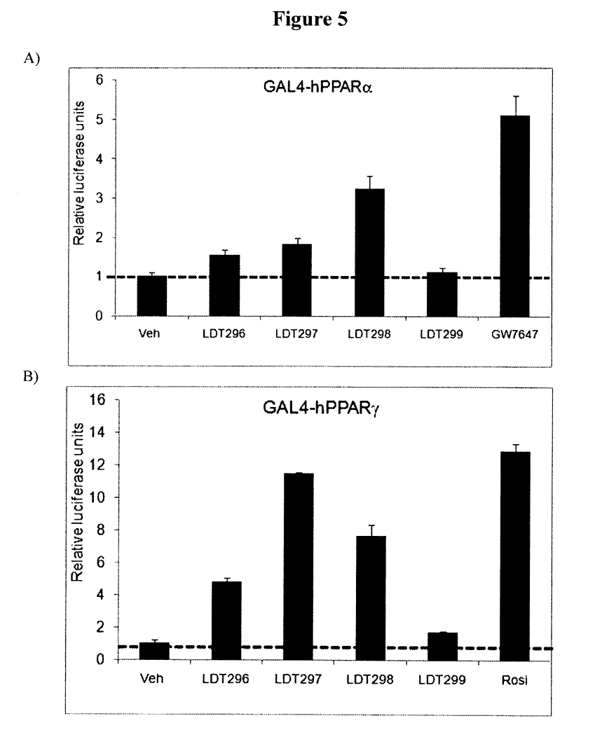

To identify PPARα and PPARγ agonists of the compounds of the Formula (II), the compounds were analyzed for activity using the GAL4-hPPARα/γ-UAS-luciferase reporter system in transiently transfected HEK293 cells as shown in FIG. 5. LDT297 showed ˜2-fold increase in activity as compared to Veh in the PPARα activation assays (FIG. 5A), whereas in the PPARγ activation assay it had a ˜12-fold increase in reporter activity. LDT298 showed comparable luciferase activation in both PPARα and PPARγ assays.

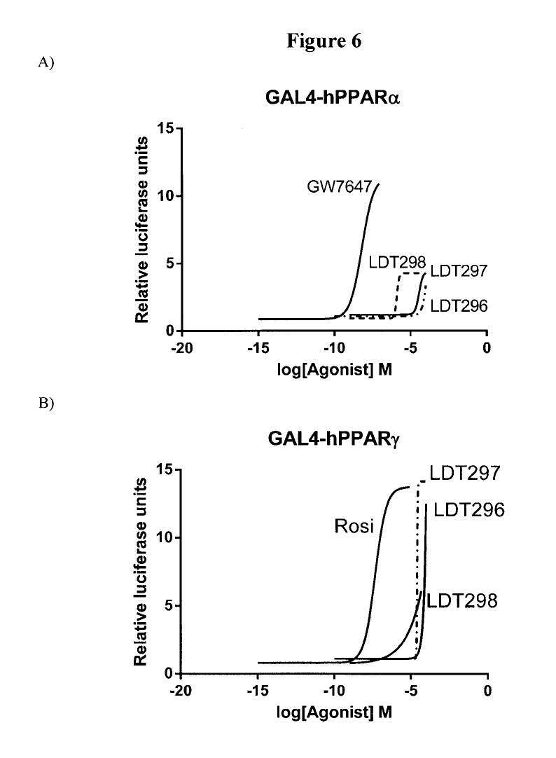

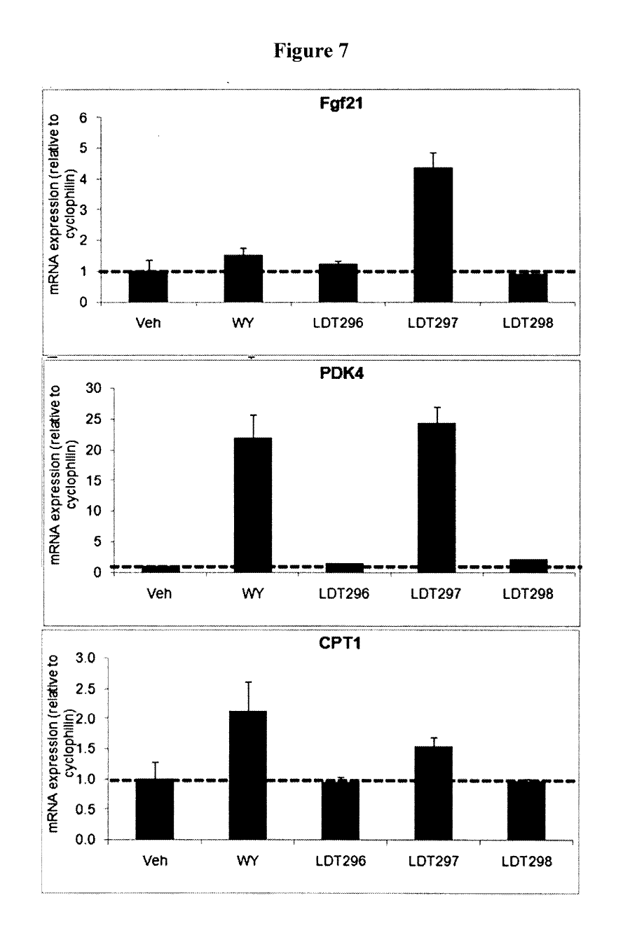

To determine the individual potencies of compounds of the Formula (II), dose-response analyses were carried out in the HEK293 cells as shown in FIG. 6. LDT296, LDT297 and LDT298 were found to be less potent partial PPARα agonists, when compared to the full agonist GW7647 (FIG. 6A). The EC50 value for LDT296 was not determined, however, LDT297 and LDT298 had EC50 values of 36 μM and 1.3 μM, respectively. Interestingly, LDT297 and LDT296 had a less potent, full agonist profile on the PPARγ receptor (FIG. 6B) when compared to the rosiglitazone control. Using curve-fitting, we estimate that the EC50 for LDT297 is 26 μM.

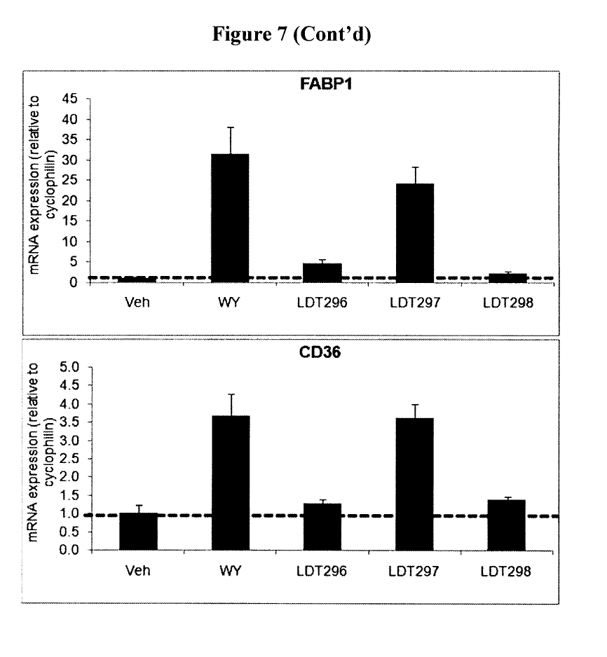

To examine the ability of compounds of the Formula (II) to upregulate the expression of PPARα target genes, primary mouse hepatocytes were treated with 50 μM of selected compounds as shown in FIG. 7. LDT297 showed upregulation of Fgf21 expression, a hormone which has potent anti-diabetic properties by promoting fatty acid oxidation, insulin sensitivity and enhanced energy expenditure. The expression of CPT-1, a rate limiting enzyme in β-oxidation, was also increased following LDT297 treatment. Moreover, the expression of PDK4, an enzyme which allows for preferential utilization of fatty acids over glucose as an energy source, was also upregulated. Finally, LDT297 treatment upregulated the expression of genes involved in fatty acid uptake, FABP1 and CD36. Overall, these results indicate that compounds of the Formula (II) (LDT297) can promote fatty acid uptake and oxidation in the liver which represent beneficial effects of PPARα activation for treatment of hyperlipidemia.

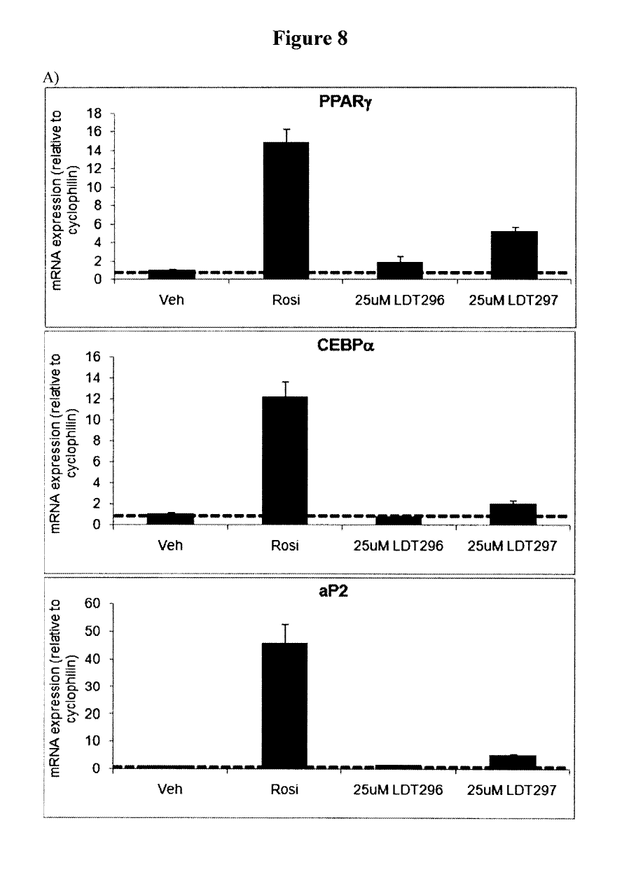

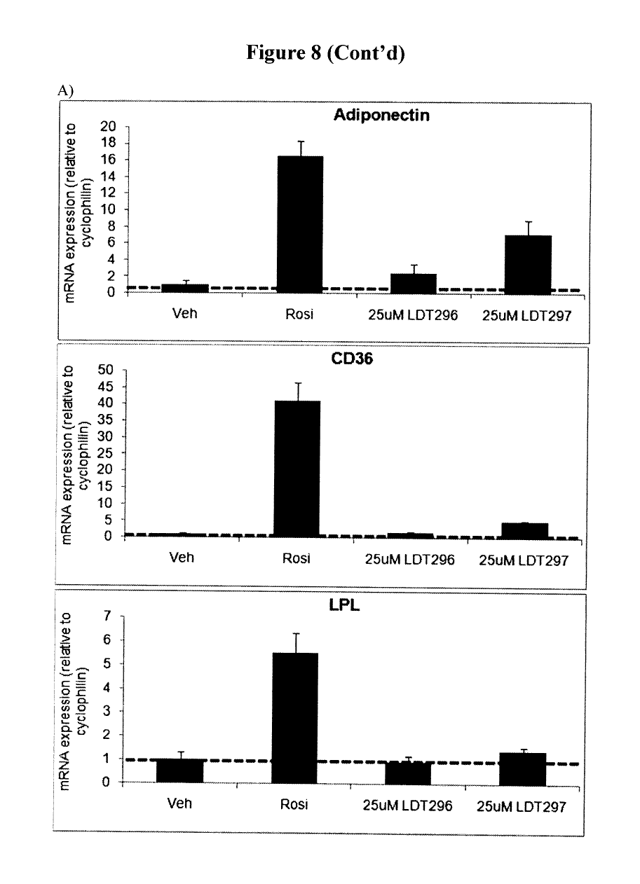

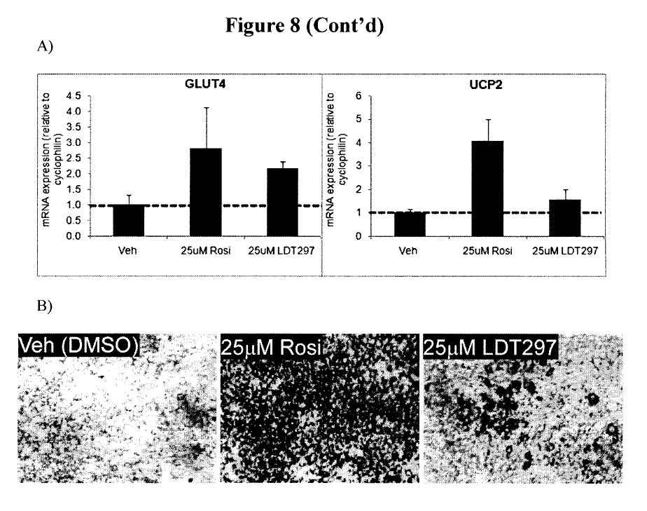

To examine the ability of compounds of the Formula (II) to upregulate the expression of PPARγ target genes, 3T3-L1 pre-adipocytes were treated with 25 μM of selected LDT compounds and monitored for differentiation as shown in FIG. 8. Similar to the compounds of the Formula (I), LDT297 upregulated the expression of PPARγ and CEBPα (master regulators of adipocyte differentiation), and adiponectin (a hormone secreted by mature adipocytes and improves insulin sensitivity) and GLUT4 (a glucose uptake transporter) (FIG. 8A). Moreover, LDT297 treatment increased the expression of genes involved in fatty acid uptake (LPL, CD36 and AP2), facilitating fatty acid oxidation. The expression of UCP2, an uncoupling protein which plays a role in increasing energy expenditure, was also upregulated by LDT297. (FIG. 8A). In line with the gene expression data, Oil red 0 staining (FIG. 8B) showed that LDT297 was able to induce adipocyte differentiation but this induction was attenuated compared to that of rosiglitazone. Overall, these results indicate that compounds of the Formula (II) (LDT297) possess the beneficial effects of PPARγ activation by showing potent increases in adiponectin levels with an overall lower adipocyte differentiation rate, which indicates the compounds can be therapeutically beneficial and permit insulin sensitization without the undesirable weight gain that occurs with TZD therapy

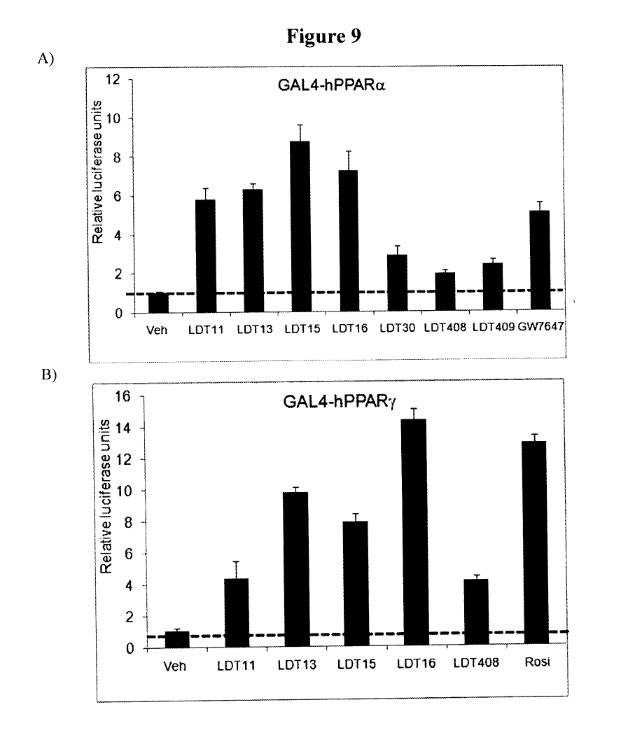

To identify novel PPARα and PPARγ agonists from the compounds of the Formula (III), the compounds were analyzed for activity using the GAL4-hPPAR/γ-UAS-luciferase reporter system as shown in Figure. All compounds in this series were active against PPARα (FIG. 9A) and exhibited 2-8-fold induction relative to vehicle control when dosed at 50 μM. Similarly, the compounds in this series were also active against PPARγ (FIG. 9B) and achieved between a 4-14-fold induction relative to vehicle control when dosed at 50 μM.

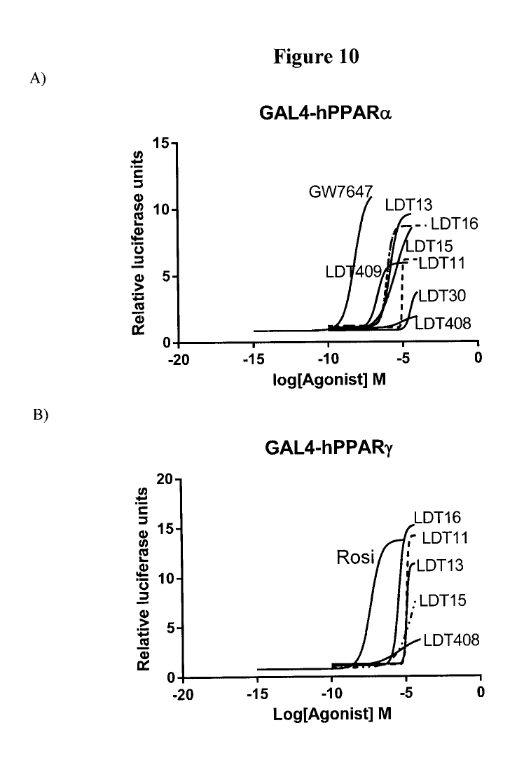

To determine the individual potencies of the compounds of the Formula (III), dose-response analyses were carried out in the HEK293 cells as shown in FIG. 10. All of the compounds were found to be less potent, partial PPARα agonists when compared to the full agonist GW7647 (FIG. 10A). The following EC50 values were obtained for PPARα: LDT11 9 μM; LDT13 7 μM; LDT15 3.5 μM; LDT16 0.9 μM; LDT30 32 μM; LDT409 0.2 μM. Similarly, LDT13, LDT15 and LDT408 were partial PPARγ agonists, whereas LDT11 and LDT16 exhibited full agonist profile compared to rosiglitazone against the PPARγ receptor (FIG. 10B). The following EC50 values were obtained for PPARγ: LDT11 12 μM; LDT13 12 μM; LDT15 42 μM; LDT16 3.6 μM.

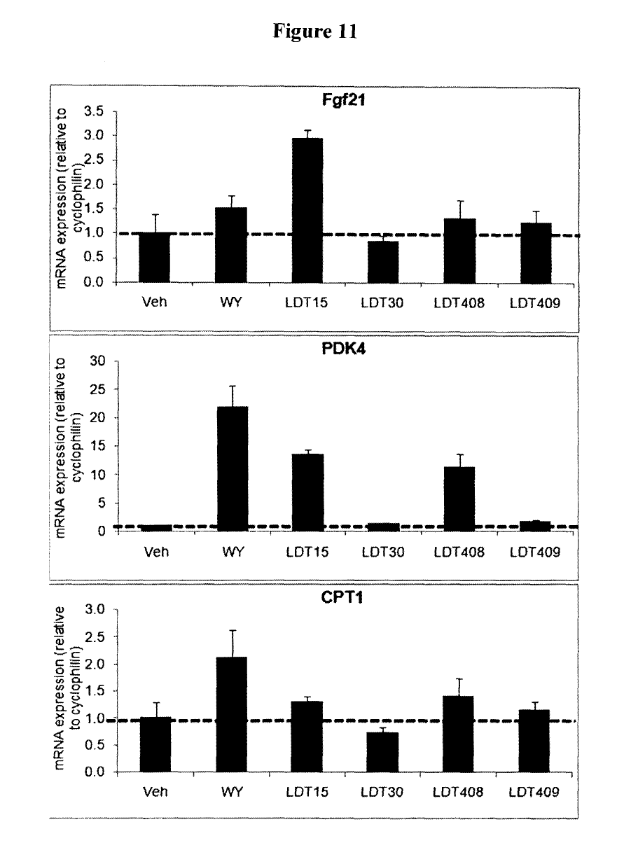

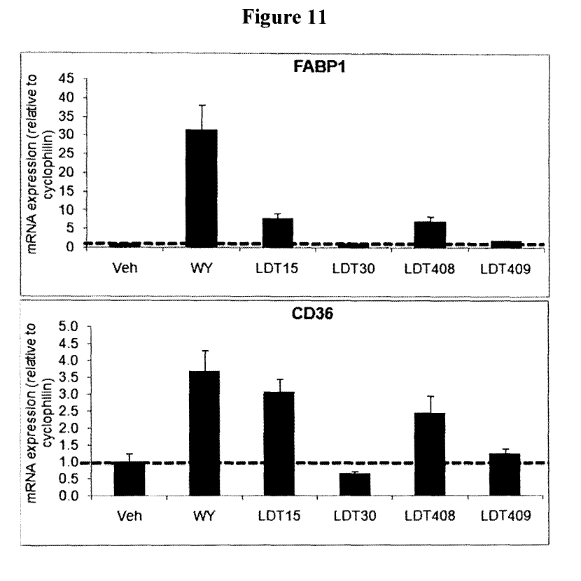

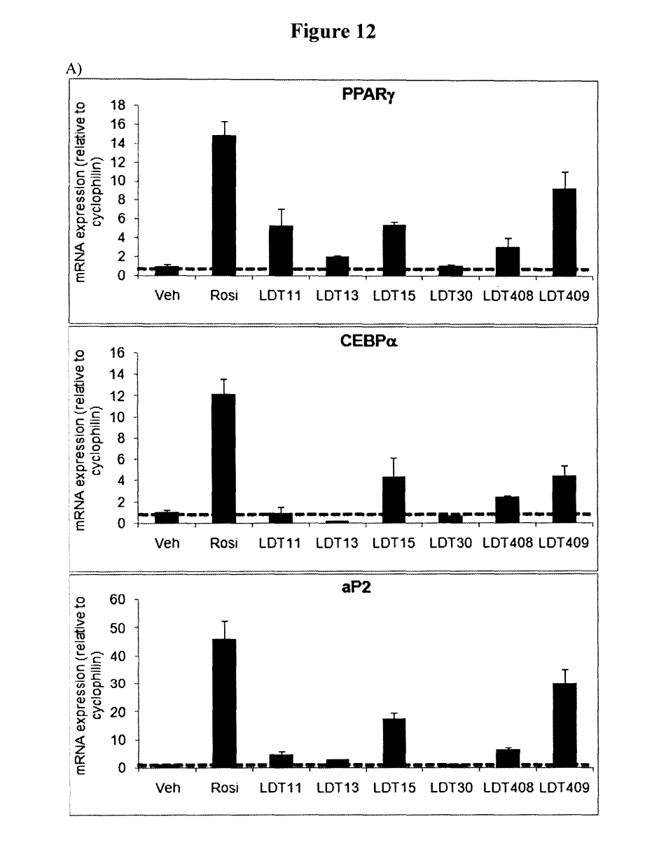

To examine the ability of the compounds of the Formula (III) to upregulate the expression of PPARα target genes, primary mouse hepatocytes were treated with 50 μM of selected compounds, as shown in FIG. 11. LDT15 showed upregulation of Fgf21 expression, a hormone which has potent anti-diabetic properties by promoting fatty acid oxidation, energy expenditure, and insulin sensitization. The expression of PDK4, an enzyme which allows for preferential utilization of fatty acids as the energy source, was upregulated by LDT15 and LDT408. FABP1 and CD36, genes important in fatty acid uptake, were also significantly increased with LDT15 and LDT408. Overall, these results indicate that the compounds can promote fatty acid uptake and oxidation in the liver that would lead to decreased circulating lipids.

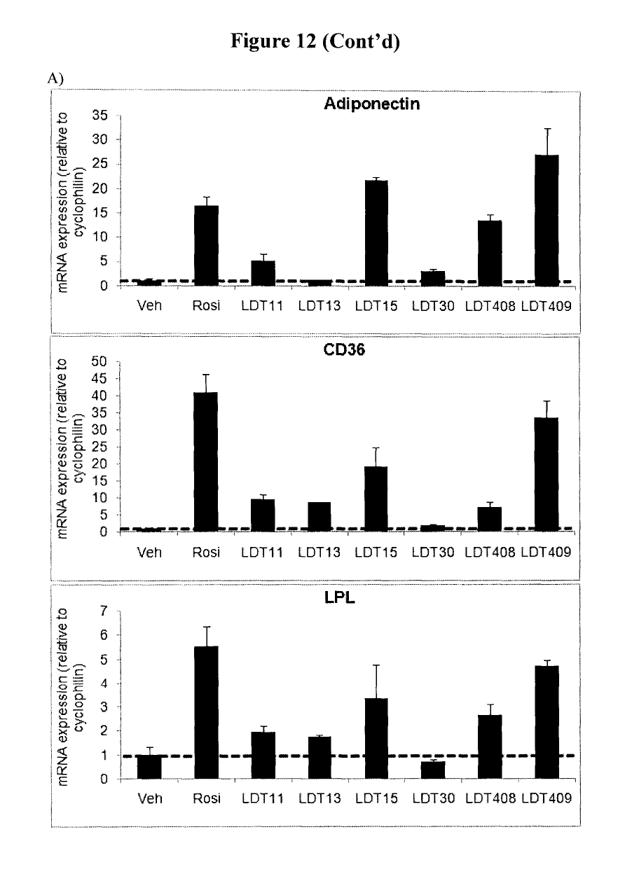

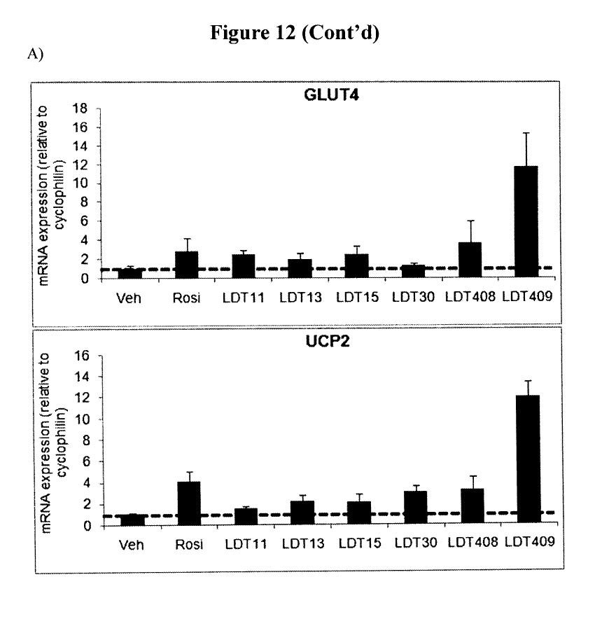

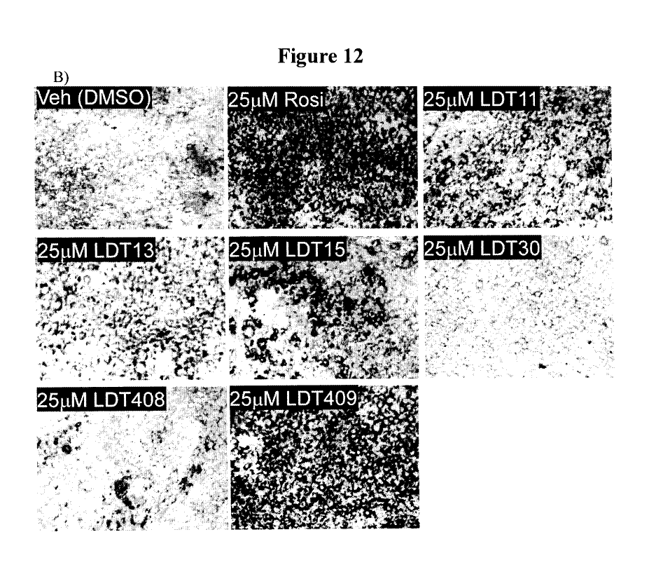

To examine the ability of the compounds of the Formula (III) to upregulate the expression of PPARγ target genes, 3T3-L1 pre-adipocytes were treated with 25 μM of selected compounds and monitored for adipocyte differentiation, as shown in FIG. 12. Most of the compounds increased the expression of PPARγ and CEBPα (master regulators of adipocyte differentiation). Adiponectin expression was induced to a similar degree as Rosi by LDT15 and LDT409; LDT11 and LDT408 also significantly increased its expression (FIG. 12A). Moreover, LDT C15 compounds increased the expression of LPL, CD36 and AP2, all of which promote lipid oxidation in fat through coordinating the uptake of fatty acids. UCP2, an uncoupling protein which promotes energy expenditure, was also upregulated by LDT15, 30, 408 and 409. GLUT4 expression was also augmented by LDT 11, 13, 15, 408 and 409. Since GLUT4 is a predominant glucose uptake transporter in adipocytes, it is possible that LDT C15 series of compounds may exert beneficial effects on hyperglycemia and insulin resistance by increasing the expression of GLUT4 in adipose tissue. Oil red 0 staining (FIG. 12B) showed that LDT409 was comparable to Rosi in its ability to induce adipocyte differentiation. In contrast, LDT15, LDT408 and LDT11 were less adipogenic compared to Rosi. These data, taken together, indicate that the compounds can serve as insulin sensitizers that are devoid the negative side effects found with TZDs.

### Example 3: Prophetic In Vivo Experiment

To test the ability of the LDT compounds to lower hyperlipidemia, lower hyperglycemia and/or increase insulin sensitivity without concomitant weight gain we will test the compounds in three animal models of metabolic disease: i) Diet-induced obese (DIO) mice, ii) leptin receptor deficient mice (db/db) and iii) Zucker diabetic fatty rats. Each model will be treated with LDT compounds for up to 6 weeks. Oral glucose tolerance and insulin tolerance tests will be administered after 4.5 and 5.5 weeks of drug treatment. DIO mice will be generated by feeding 6 week old C57BI/6 mice a high fat high cholesterol diet for 12 weeks. Drug treatment will be initiated when the mice are 18 weeks old and be administered daily for six additional weeks. Body composition (body weight, fat distribution, lean mass), total energy expenditure, gene expression, histologic analysis, and plasma analyses will be performed at the end of the study. Samples will be processed to assess insulin sensitivity and hyperlipidemia by measuring plasma levels of FFAs, glucose, insulin, triglycerides, cholesterol, and FGF21. Tissue cholesterol and triglyceride levels will also be measured. Tissues will be processed for gene and protein analysis of PPAR target genes.

To test the ability of the LDT compounds to mitigate the progression of neurodegenerative diseases, we will test the compounds in an animal model of Alzheimer's disease (TgCRND8 mice). Wildtype and TgCRND8 mice will be treated with LDT compounds by daily administration for up to 8 weeks. Fear conditioning testing will be performed at the end of the study to assess whether any improvements in cognitive function were detected. After sacrificing the mice, the brains will be processed for Aβ1-40 and Aβ1-42 quantitation. Intracerebral levels of PPAR target genes will be measured by real-time PCR and Western blotting. Immunohistochemistry will be performed to assess amyloid plaque burden.

### REFERENCES CITED HEREIN

1. International Diabetes Federation. 2014; http://www.idf.org/metabolic-syndrome. Accessed May 15, 2014.
2. Eckel R H, Alberti K G, Grundy S M, Zimmet P Z. The metabolic syndrome. Lancet. 2010; 375(9710):181-183.
3. Evans R M, Barish G D, Wang Y X. PPARs and the complex journey to obesity. Nature medicine. 2004; 10(4):355-361.
4. Kersten S, Desvergne B, Wahli W. Roles of PPARs in health and disease. Nature. 2000; 405(6785):421-424.
5. Fruchart J C. Peroxisome proliferator-activated receptor-alpha (PPARalpha): at the crossroads of obesity, diabetes and cardiovascular disease. Atherosclerosis. 2009; 205(1): 1-8.
6. Stienstra R, Mandard S, Patsouris D, Maass C, Kersten S, Muller M. Peroxisome proliferator-activated receptor alpha protects against obesity-induced hepatic inflammation. Endocrinology. 2007; 148(6):2753-2763.
7. Daynes R A, Jones D C. Emerging roles of PPARs in inflammation and immunity. Nat Rev Immunol. 2002; 2(10):748-759.
8. Inagaki T, Dutchak P, Zhao G, Ding X, Gautron L, Parameswara V, Li Y, Goetz R, Mohammadi M, Esser V, Elmquist J K, Gerard R D, Burgess S C, Hammer R E, Mangelsdorf D J, Kliewer S A. Endocrine regulation of the fasting response by PPARalpha-mediated induction of fibroblast growth factor 21. Cell metabolism. 2007; 5(6):415-425.
9. Oishi K, Uchida D, Ishida N. Circadian expression of FGF21 is induced by PPARalpha activation in the mouse liver. FEBS letters. 2008; 582(25-26):3639-3642.
10. Gimeno R E, Moller D E. FGF21-based pharmacotherapy—potential utility for metabolic disorders. Trends in endocrinology and metabolism: TEM. 2014.
11. Domouzoglou E M, Maratos-Flier E. Fibroblast growth factor 21 is a metabolic regulator that plays a role in the adaptation to ketosis. The American journal of clinical nutrition. 2011; 93(4): 901S-905.
12. Gaich G, Chien J Y, Fu H, Glass L C, Deeg M A, Holland W L, Kharitonenkov A, Bumol T, Schilske H K, Moller D E. The effects of LY2405319, an FGF21 analog, in obese human subjects with type 2 diabetes. Cell metabolism. 2013; 18(3): 333-340.
13. Potthoff M J, Inagaki T, Satapati S, Ding X, He T, Goetz R, Mohammadi M, Finck B N, Mangelsdorf D J, Kliewer S A, Burgess S C. FGF21 induces PGC-1alpha and regulates carbohydrate and fatty acid metabolism during the adaptive starvation response. Proceedings of the National Academy of Sciences of the United States of America. 2009; 106(26): 10853-10858.
14. Mashili F L, Austin R L, Deshmukh A S, Fritz T, Caidahl K, Bergdahl K, Zierath J R, Chibalin A V, Moller D E, Kharitonenkov A, Krook A. Direct effects of FGF21 on glucose uptake in human skeletal muscle: implications for type 2 diabetes and obesity. Diabetes Metab Res Rev. 2011; 27(3):286-297.
15. Arner P, Pettersson A, Mitchell P J, Dunbar J D, Kharitonenkov A, Ryden M. FGF21 attenuates lipolysis in human adipocytes—a possible link to improved insulin sensitivity. FEBS letters. 2008; 582(12):1725-1730.
16. Fisher F M, Estall J L, Adams A C, Antonellis P J, Bina H A, Flier J S, Kharitonenkov A, Spiegelman B M, Maratos-Flier E. Integrated regulation of hepatic metabolism by fibroblast growth factor 21 (FGF21) in vivo. Endocrinology. 2011; 152(8):2996-3004.
17. Xu J, Stanislaus S, Chinookoswong N, Lau Y Y, Hager T, Patel J, Ge H, Weiszmann J, Lu S C, Graham M, Busby J, Hecht R, Li Y S, Li Y, Lindberg R, Veniant M M. Acute glucose-lowering and insulin-sensitizing action of FGF21 in insulin-resistant mouse models-association with liver and adipose tissue effects. American journal of physiology Endocrinology and metabolism. 2009; 297(5): E1105-1114.
18. Lee P, Linderman J D, Smith S, Brychta R J, Wang J, Idelson C, Perron R M, Werner C D, Phan G Q, Kammula U S, Kebebew E, Pacak K, Chen K Y, Celi F S. Irisin and FGF21 are cold-induced endocrine activators of brown fat function in humans. Cell metabolism. 2014; 19(2):302-309.
19. Bookout A L, de Groot M H, Owen B M, Lee S, Gautron L, Lawrence H L, Ding X, Elmquist J K, Takahashi J S, Mangelsdorf D J, Kliewer S A. FGF21 regulates metabolism and circadian behavior by acting on the nervous system. Nature medicine. 2013; 19(9): 1147-1152.
20. Fisher F M, Kleiner S, Douris N, Fox E C, Mepani R J, Verdeguer F, Wu J, Kharitonenkov A, Flier J S, Maratos-Flier E, Spiegelman B M. FGF21 regulates PGC-1alpha and browning of white adipose tissues in adaptive thermogenesis. Genes & development. 2012; 26(3):271-281.
21. Kintscher U, Law R E. PPARgamma-mediated insulin sensitization: the importance of fat versus muscle. American journal of physiology Endocrinology and metabolism. 2005; 288(2): E287-291.
22. Way J M, Harrington W W, Brown K K, Gottschalk W K, Sundseth S S, Mansfield T A, Ramachandran R K, Willson T M, Kliewer S A. Comprehensive messenger ribonucleic acid profiling reveals that peroxisome proliferator-activated receptor gamma activation has coordinate effects on gene expression in multiple insulinsensitive tissues. Endocrinology. 2001; 142(3): 1269-1277.
23. Willson T M, Lambert M H, Kliewer S A. Peroxisome proliferator-activated receptor gamma and metabolic disease. Annu Rev Biochem. 2001; 70:341-367.
24. Boden G, Homko C, Mozzoli M, Showe L C, Nichols C, Cheung P. Thiazolidinediones upregulate fatty acid uptake and oxidation in adipose tissue of diabetic patients. Diabetes. 2005; 54(3):880-885.
25. Sugii S, Olson P, Sears D D, Saberi M, Atkins A R, Barish G D, Hong S H, Castro G L, Yin Y Q, Nelson M C, Hsiao G, Greaves D R, Downes M, Yu R T, Olefsky J M, Evans R M. PPARgamma activation in adipocytes is sufficient for systemic insulin sensitization. Proceedings of the National Academy of Sciences of the United States of America. 2009; 106(52):22504-22509.
26. Ahmadian M, Suh J M, Hah N, Liddle C, Atkins A R, Downes M, Evans R M. PPARgamma signaling and metabolism: the good, the bad and the future. Nature medicine. 2013; 19(5): 557-566.
27. Lin Z, Tian H, Lam K S, Lin S, Hoo R C, Konishi M, Itoh N, Wang Y, Bornstein S R, Xu A, Li X. Adiponectin mediates the metabolic effects of FGF21 on glucose homeostasis and insulin sensitivity in mice. Cell metabolism. 2013; 17(5):779-789.
28. Holland W L, Adams A C, Brozinick J T, Bui H H, Miyauchi Y, Kusminski C M, Bauer S M, Wade M, Singhal E, Cheng C C, Volk K, Kuo M S, Gordillo R, Kharitonenkov A, Scherer P E. An FGF21-adiponectin-ceramide axis controls energy expenditure and insulin action in mice. Cell metabolism. 2013; 17(5): 790-797.
29. Nawrocki A R, Rajala M W, Tomas E, Pajvani U B, Saha A K, Trumbauer M E, Pang Z, Chen A S, Ruderman N B, Chen H, Rossetti L, Scherer P E. Mice lacking adiponectin show decreased hepatic insulin sensitivity and reduced responsiveness to peroxisome proliferator-activated receptor gamma agonists. The Journal of biological chemistry. 2006; 281(5):2654-2660.
30. Okuno A, Tamemoto H, Tobe K, Ueki K, Mori Y, Iwamoto K, Umesono K, Akanuma Y, Fujiwara T, Horikoshi H, Yazaki Y, Kadowaki T. Troglitazone increases the number of small adipocytes without the change of white adipose tissue mass in obese Zucker rats. The Journal of clinical investigation. 1998; 101(6):1354-1361.
31. Chawla A, Barak Y, Nagy L, Liao D, Tontonoz P, Evans R M. PPAR-gamma dependent and independent effects on macrophage-gene expression in lipid metabolism and inflammation. Nature medicine. 2001; 7(1):48-52.
32. Odegaard J I, Ricardo-Gonzalez R R, Goforth M H, Morel C R, Subramanian V, Mukundan L, Red Eagle A, Vats D, Brombacher F, Ferrante A W, Chawla A. Macrophage-specific PPARgamma controls alternative activation and improves insulin resistance. Nature. 2007; 447(7148):1116-1120.
33. Kung J, Henry R R. Thiazolidinedione safety. Expert Opin Drug Saf. 2012; 11(4):565-579.
34. Observation of an Increased Incidence of Fractures in Female Patients who Received Long-Term Treatment With Actos (Pioglitazone HCl) Tablets for Type 2 Diabetes Mellitus. Deerfield, Ill. Takeda Pharmaceuticals North America.
35. Kahn S E, Zinman B, Lachin J M, Haffner S M, Herman W H, Holman R R, Kravitz B G, Yu D, Heise M A, Aftring R P, Viberti G, Diabetes Outcome Progression Trial Study G. Rosiglitazone-associated fractures in type 2 diabetes: an Analysis from A Diabetes Outcome Progression Trial (ADOPT). Diabetes Care. 2008; 31(5):845-851.
36.http://www.fda.gov/downloads/Drugs/DrugSafety/PostmarketDrugSafetylnf ormationforPatientsandProviders/UCM143413.pdf. Accessed May 20, 2014.
37. Bortolini M, Wright M B, Bopst M, Balas B. Examining the safety of PPAR agonists—current trends and future prospects. Expert Opin Drug Saf. 2013; 12(1):65-79.
38. Hiukka A, Maranghi M, Matikainen N, Taskinen M R. PPARalpha: an emerging therapeutic target in diabetic microvascular damage. Nat Rev Endocrinol. 2010; 6(8):454-463.
39. Gregoire F M, Zhang F, Clarke H J, Gustafson T A, Sears D D, Favelyukis S, Lenhard J, Rentzeperis D, Clemens L E, Mu Y, Lavan B E. MBX-102/JNJ39659100, a novel peroxisome proliferator-activated receptor-ligand with weak transactivation activity retains antidiabetic properties in the absence of weight gain and edema. Molecular endocrinology. 2009; 23(7):975-988.
40. Fukui Y, Masui S, Osada S, Umesono K, Motojima K. A new thiazolidinedione, NC-2100, which is a weak PPAR-gamma activator, exhibits potent antidiabetic effects and induces uncoupling protein 1 in white adipose tissue of KKAy obese mice. Diabetes. 2000; 49(5):759-767.
40a. Pochetti G, Mitro N, Lavecchia A, Gilardi F, Besker N, Scotti E, Aschi M, Re N, Fracchiolla G, Laghezza A, Tortorella P, Montanari R, Novellino E, Mazza F, Crestani M, Loiodice F. Structural insight into peroxisome proliferator-activated receptor gamma binding of two ureidofibrate-like enantiomers by molecular dynamics, cofactor interaction analysis, and site-directed mutagenesis. J Med Chem. 2010; 53(11):4354-4366.
41. Ren Y. Peroxisome Proliferator-Activator Receptor gamma: A Link between Macrophage CD36 and Inflammation in Malaria Infection. PPAR Res. 2012; 2012:640769.
42. Balachandar S, Katyal A. Peroxisome proliferator activating receptor (PPAR) in cerebral malaria (C M): a novel target for an additional therapy. Eur J Clin Microbiol Infect Dis. 2011; 30(4):483-498.
43. Boggild A K, Krudsood S, Patel S N, Serghides L, Tangpukdee N, Katz K, Wilairatana P, Liles W C, Looareesuwan S, Kain K C. Use of peroxisome proliferator-activated receptor gamma agonists as adjunctive treatment for Plasmodium falciparum malaria: a randomized, double-blind, placebo-controlled trial. Clin Infect Dis. 2009; 49(6):841-849.
44. Serghides L, Kain K C. Peroxisome proliferator-activated receptor gamma-retinoid X receptor agonists increase CD36-dependent phagocytosis of Plasmodium falciparum-parasitized erythrocytes and decrease malaria-induced TNF-alpha secretion by monocytes/macrophages. Journal of immunology. 2001; 166(11):6742-6748.
45. Moreira R O, Campos S C, Soldera A L. Type 2 Diabetes Mellitus and Alzheimer's Disease: from physiopathology to treatment implications. Diabetes Metab Res Rev. 2013.
46. Ferreira S T, Clarke J R, Bomfim T R, De Felice F G. Inflammation, defective insulin signaling, and neuronal dysfunction in Alzheimer's disease. Alzheimers Dement. 2014; 10(1 Suppl):S76-83.
47. Zhou W, Hu W. Anti-neuroinflammatory agents for the treatment of Alzheimer's disease. Future Med Chem. 2013; 5(13):1559-1571.
48. Combs C K, Johnson D E, Karlo J C, Cannady S B, Landreth G E. Inflammatory mechanisms in Alzheimer's disease: inhibition of beta-amyloid-stimulated proinflammatory responses and neurotoxicity by PPARgamma agonists. J Neurosci. 2000; 20(2):558-567.
49. McGilvray I D, Serghides L, Kapus A, Rotstein O D, Kain K C. Nonopsonic monocyte/macrophage phagocytosis of Plasmodium falciparum-parasitized erythrocytes: a role for CD36 in malarial clearance. Blood. 2000; 96(9):3231-3240.
50. Patel S N, Lu Z, Ayi K, Serghides L, Gowda D C, Kain K C. Disruption of CD36 impairs cytokine response to Plasmodium falciparum glycosylphosphatidylinositol and confers susceptibility to severe and fatal malaria in vivo. Journal of immunology. 2007; 178(6):3954-3961.
51. Patel S N, Serghides L, Smith T G, Febbraio M, Silverstein R L, Kurtz T W, Pravenec M, Kain K C. CD36 mediates the phagocytosis of Plasmodium falciparum-infected erythrocytes by rodent macrophages. J Infect Dis. 2004; 189(2):204-213.
52. Chen Z, Zhong C. Decoding Alzheimer's disease from perturbed cerebral glucose metabolism: implications for diagnostic and therapeutic strategies. Prog Neurobiol. 2013; 108:21-43.
53. Correia S C, Santos R X, Carvalho C, Cardoso S, Candeias E, Santos M S, Oliveira C R, Moreira P I. Insulin signaling, glucose metabolism and mitochondria: major players in Alzheimer's disease and diabetes interrelation. Brain Res. 2012; 1441:64-78.
53a. Ott A, Stolk R P, van Harskamp F, Pols H A, Hofman A, Breteler M M. Diabetes mellitus and the risk of dementia: The Rotterdam Study. Neurology. 1999; 53(9): 1937-1942.
53b. Janson J, Laedtke T, Parisi J E, O'Brien P, Petersen R C, Butler P C. Increased risk of type 2 diabetes in Alzheimer disease. Diabetes. 2004; 53(2):474-481.
54. Tanzi R E, Bertram L. Twenty years of the Alzheimer's disease amyloid hypothesis: a genetic perspective. Cell. 2005; 120(4):545-555.
54a. Clodfelder-Miller B J, Zmijewska A A, Johnson G V, Jope R S. Tau is hyperphosphorylated at multiple sites in mouse brain in vivo after streptozotocin-induced insulin deficiency. Diabetes. 2006; 55(12):3320-3325.
55. Mandrekar-Colucci S, Landreth G E. Nuclear receptors as therapeutic targets for Alzheimer's disease. Expert Opin Ther Targets. 2011; 15(9):1085-1097.
55a. Yamanaka M, Ishikawa T, Griep A, Axt D, Kummer M P, Heneka M T. PPARgamma/RXRalpha-induced and CD36-mediated microglial amyloid-beta phagocytosis results in cognitive improvement in amyloid precursor protein/presenilin 1 mice. J Neurosci. 2012; 32(48):17321-17331.
55b. Deane R, Sagare A, Hamm K, Parisi M, Lane S, Finn M B, Holtzman D M, Zlokovic B V. apoE isoform-specific disruption of amyloid beta peptide clearance from mouse brain. The Journal of clinical investigation. 2008; 118(12):4002-4013.
56. Cramer P E, Cirrito J R, Wesson D W, Lee C Y, Karlo J C, Zinn A E, Casali B T, Restivo J L, Goebel W D, James M J, Brunden K R, Wilson D A, Landreth G E. ApoE-directed therapeutics rapidly clear beta-amyloid and reverse deficits in A D mouse models. Science. 2012; 335(6075):1503-1506.
57. Wang R, Li J J, Diao S, Kwak Y D, Liu L, Zhi L, Bueler H, Bhat N R, Williams R W, Park E A, Liao F F. Metabolic stress modulates Alzheimer's beta-secretase gene transcription via SIRT1-PPARgamma-PGC-1 in neurons. Cell metabolism. 2013; 17(5):685-694.
58. Nenov M N, Laezza F, Haidacher S J, Zhao Y, Sadygov R G, Starkey J M, Spratt H, Luxon B A, Dineley K T, Denner L. Cognitive enhancing treatment with a PPARgamma agonist normalizes dentate granule cell presynaptic function in Tg2576 APP mice. J Neurosci. 2014; 34(3):1028-1036.
59. Denner L A, Rodriguez-Rivera J, Haidacher S J, Jahrling J B, Carmical J R, Hernandez C M, Zhao Y, Sadygov R G, Starkey J M, Spratt H, Luxon B A, Wood T G, Dineley K T. Cognitive enhancement with rosiglitazone links the hippocampal PPARgamma and ERK MAPK signaling pathways. J Neurosci. 2012; 32(47):16725-16735a.
60. Rodriguez-Rivera J, Denner L, Dineley K T. Rosiglitazone reversal of Tg2576 cognitive deficits is independent of peripheral gluco-regulatory status. Behav Brain Res. 2011; 216(1):255-261.
61. Xu S, Liu G, Bao X, Wu J, Li S, Zheng B, Anwyl R, Wang Q. Rosiglitazone prevents amyloid-beta oligomer-induced impairment of synapse formation and plasticity via increasing dendrite and spine mitochondrial number. J Alzheimers Dis. 2014; 39(2):239-251.
62. Risner M E, Saunders A M, Altman J F, Ormandy G C, Craft S, Foley I M, Zvartau-Hind M E, Hosford D A, Roses A D, Rosiglitazone in Alzheimer's Disease Study G. Efficacy of rosiglitazone in a genetically defined population with mild-to-moderate Alzheimer's disease. Pharmacogenomics J. 2006; 6(4):246-254.
63. Watson G S, Cholerton B A, Reger M A, Baker L D, Plymate S R, Asthana S, Fishel M A, Kulstad J J, Green P S, Cook D G, Kahn S E, Keeling M L, Craft S.
Preserved cognition in patients with early Alzheimer disease and amnestic mild cognitive impairment during treatment with rosiglitazone: a preliminary study. Am J Geriatr Psychiatry. 2005; 13(11):950-958.
64. Sato T, Hanyu H, Hirao K, Kanetaka H, Sakurai H, Iwamoto T. Efficacy of PPARgamma agonist pioglitazone in mild Alzheimer disease. Neurobiol Aging. 2011; 32(9):1626-1633.

1. A compound of the Formula (Ia) 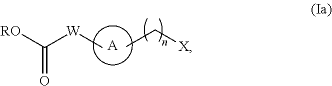
wherein
Ring A is
(i) optionally substituted phenyl, or
(ii) optionally substituted (C₅-C₆)-heteroaryl,
in which the optional substituents are selected from one to four of halo, OH, O—(C₁-C₆)-alkyl, (C₁-C₆)-alkyl, —C(O)OH, —OC(O)—(C₁-C₆)-alkyl or —C(O)O—(C₁-C₆)-alkyl;
X is
(i) R′,
(ii) —OR′,
(iii) —C(O)O—R′,
(iv) —C(O)—R′, or
(v) —C(O)—NR′R″;
R′ and R″ are each independently or simultaneously
(i) H,
(ii) (C₁-C₈)-alkyl optionally substituted by (C₆-C₁₀)aryl, or
(iii) (C₆-C₁₀)-aryl optionally substituted by (C₁-C₈)alkyl or (C₆-C₁₀)aryl,
W is (C₂-C₆)-alkylene, wherein
(ii.a) at least one carbon atom of the (C₂-C₆)-alkylene group is substituted with R₁ and/or R₂; and
(ii.b) at least one carbon atom of the (C₂-C₆)-alkylene group is replaced with an oxygen atom;
R₁ is
(i) H,
(ii) (C₁-C₈)-alkyl, or
(iii) (C₃-C₈)-cycloalkyl,
R₂ is
(i) H,
(ii) (C₁-C₈)-alkyl, or
(iii) (C₃-C₈)-cycloalkyl, or
R₁ and R₂ taken together form a (C₃-C₈)-cycloalkyl ring,
R is H or (C₁-C₈)-alkyl, and
n is 6, 7, 8, 9, or 10,
or a pharmaceutically acceptable salt, solvate, prodrug and/or stereoisomer thereof.

2. The compound of the Formula (Ia) according to claim 1, wherein Ring A is optionally substituted phenyl.

3. The compound of the Formula (Ia) according to claim 1, wherein Ring A is optionally substituted once with OH, O—(C₁-C₆)-alkyl, —C(O)OH, —OC(O)—(C₁-C₆)-alkyl or —C(O)O—(C₁-C₆)-alkyl.

4. The compound of the Formula (Ia) according to claim 1, wherein Ring A has the following structure

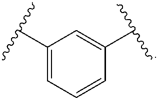 [SMILES: *c1cccc(*)c1]

5. (canceled)

6. The compound of the Formula (Ia) according to claim 1, wherein X is R′, —OR′, —C(O)O—R′, pr —C(O)—R′, wherein R′ H, (C₁-C₈)-alkyl or (C₆-C₁₀)-aryl.

7. The compound of the Formula (Ia) according to claim 6, wherein X is OR′ or —C(O)O—R′, wherein R′ H or (C₁-C₈)-alkyl.

8. The compound of the Formula (Ia) according to claim 7, wherein X is OH, —C(O)OH or —C(O)O—(C₁-C₈)-alkyl.

9. The compound of the Formula (Ia) according to claim 8, wherein X is OH, —C(O)OH or —C(O)O—(C₁-C₄)-alkyl.

10. The compound of the Formula (Ia) according to claim 9, wherein X is OH, —C(O)OH or —C(O)OCH₂CH₃.

11. The compound of the Formula (Ia) according to claim 1, wherein W is —C(R₁)(R₂)—O— or —O—C(R₁)(R₂)—, wherein R₁ is H, (C₁-C₆)-alkyl or (C₃-C₆)-cycloalkyl, R₂ is (C₁-C₆)-alkyl or (C₃-C₆)-cycloalkyl, or R₁ and R₂ taken together form a (C₃-C₆)-alkyl ring.

12. The compound of the Formula (Ia) according to claim 11, wherein W is —C(R₁)(R₂)—O— or —O—C(R₁)(R₂)—, wherein R₁ is H, (C₁-C₃)-alkyl or (C₃-C₆)-cycloalkyl, and R₂ is (C₁-C₃)-alkyl or (C₃-C₆)-cycloalkyl.

13. The compound of the Formula (Ia) according to claim 12, wherein W is —C(CH₃)(CH₃)—O— or —O—C(CH₃)(CH₃)—.

14. The compound of the Formula (Ia) according to claim 13, wherein W is

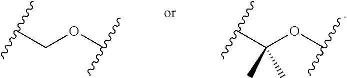

15. The compound of the Formula (Ia) according claim 1, wherein R is H or (C₁-C₆)-alkyl.

16. The compound of the Formula (Ia) according to claim 15, wherein R is H, CH₃ or CH₂CH₃.

17. The compound of the Formula (Ia) according to claim 1, wherein n is 6, 7, 8, or 9.

18. The compound of the Formula (Ia) according to claim 17, wherein n is 7 or 8.

19. The compound of the Formula (Ia) according to claim 1, wherein the compound of the Formula (Ia) is

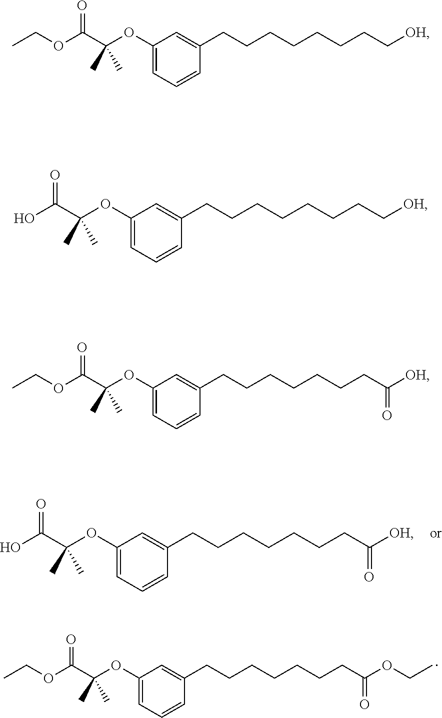

20.-47. (canceled)

48. A method for treating or preventing a disease or condition for which PPAR modulation provides a therapeutic benefit, comprising administering an effective amount of a compound of the Formula (I) according to claim 1, to a patient in need thereof.

49. The method according to claim 48, wherein the disease or condition is metabolic syndrome, obesity, hyperlipidemia, elevated fasting blood glucose, elevated blood pressure, low HDL cholesterol, type 2 diabetes, cardiovascular disease, a neurodegenerative disease, malaria or irritable bowel syndrome.

50. (canceled)

51. (canceled)
